(19) United States
(12) Patent Application Publication
(10) Pub. No.: US 2007/0167784 A1
(43) Pub. Date: Jul. 19, 2007

(54) Real-time Elastic Registration to Determine Temporal Evolution of Internal Tissues for Image-Guided Interventions

(76) Inventors: Raj Shekhar, Elkridge, MD (US); Carlos Castro-Pareja, Aloha, OR (US); Omkar Dandekar, Baltimore, MD (US)

Correspondence Address: Eugene Molinelli;Evans & Molinelli PLLC, P. O. Box 7024, Fairfax Station, VA, 22039, US

(21) Appl. No.: 11/609,458

(22) Filed: Dec. 12, 2006

Related U.S. Application Data
(60) Provisional application No. 60/749,903, filed on Dec. 13, 2005.

(51) Int. Cl. A61B 8/00

(52) U.S. Cl. 600/443

(57) ABSTRACT

Techniques for indicating arrangement of moving target tissue in a living body include receiving first scan data based at least in part on a first mode of measuring with high spatial resolution over a first duration at a first time. Also received is second scan data representing a scan of the living body based at least in part on a second mode of measuring at a second time. The second mode can be different with a second duration and a repeat rate greater than a repeat rate for the first scan data. An elastic transform is determined that registers the first scan data elastically to the second scan data. A particular spatial arrangement of the moving target tissue is indicted based on the elastic transform. These techniques can be used to update a pre-intervention plan and highlight target detail by registering pre-intervention data to second scan data during the intervention.

### CROSS-REFERENCE TO RELATED APPLICATIONS

This application claims benefit of Provisional Appln. 60/749,903, filed Dec. 13, 2005, the entire contents of which are hereby incorporated by reference as if fully set forth herein, under 35 U.S.C. § 119(e).

### STATEMENT OF GOVERNMENTAL INTEREST

This invention was made with Government support under Grant No. DAMD17-99-1-9034 and DAMD17-03-2-001 awarded by the Department of Defense. The Government has certain rights in the invention.

### BACKGROUND OF THE INVENTION

1. Field of the Invention

The present invention relates to registering one scan of internal tissues of a living body with another scan in order to combine the information in the two scans for improved treatment of the living body, such as during image-guided intervention; and in particular to using elastic registration to more accurately combine the information for more effective or less harmful treatment, or both.

2. Description of the Related Art

Healthcare delivery, whether financed by private or public funds, amounts to a multi-billion dollar business. Advances in healthcare improve the product obtained for those dollars and renders older techniques obsolete.

Several modern techniques for treatment of diseases of internal tissues of living bodies involve directing treatment to particular tissues and avoiding others. For example, in radiation therapy, one or more radioactive sources or beams of high energy particles are placed or focused on diseased tissues, such as tumors, while avoiding healthy tissues. In interventional radiology, probes are inserted into a living body to extract diseased tissue or introduce therapeutic agents at the site of particular diseased or healthy tissue in a living body. The efficacy and safety of such directed treatments are affected by the accuracy of placement of the treatment.

Several technologies are available for non-invasively measuring the arrangement of tissues within a living body. These technologies, often called imaging technologies, produce scans of spatially arranged scan elements that depict spatial variations in measured quantities that are related to spatial changes of one or more physical properties in the tissues. Often the measured quantity is intensity of electromagnetic or acoustic energy received in some time interval from some direction. The measured quantity depends on the spatial arrangement of absorption or speed in the intervening tissues, which in turn varies with the type of tissue. Well known imaging technologies includes computer-aided tomography of low intensity X-rays (CT), nuclear magnetic resonance (NMR) imaging (MRI), positron emission tomography (PET) and ultrasound (US) imaging, among others. In various arrangements, two-dimensional (2D) and three-dimensional (3D) scans are formed. Such scans are also called images. Scan elements in a 2D scan are sometimes called picture elements (pixels) and scan elements in a 3D scan are sometimes called volume elements (voxels). A treatment based on one or more such scans is called an image-guided intervention.

In stationary tissues, such as those within the skull, the spatial arrangement of the tissue is constant and well known by fixing the position of certain external skeletal features that are used as landmarks, and collecting one or more images relative to those landmarks.

However, soft tissues outside the skull are able to flex and change size, shape or position over time, even when referenced to certain skeletal features that can be fixed. For example, organs and tumors in or near the thoracic cavity ebb and flow with the breathing of the living body. Tissue in and near the heart move with the beating of the heart. Tissues near the gastrointestinal track and urinary bladder, including the bladder and prostate in the human male, swell and shrink with the amount of consumed food and fluids being processed by the living body, and by the history of physical movement of the living body between scans.

For directed treatments that are administered over times long compared to the time scales of such flexing of soft tissue, a single scan of the soft tissue, no matter how high the spatial resolution, is not accurate for the entire treatment. Thus radiation directed to a target tissue (e.g., cancerous prostate) based on a single CT scan of the prostate can lead to irradiating non-target tissue during part of the treatment time, and failing to irradiate some target tissue during part of the treatment time. Similarly, navigating a probe according to a plan based on a planning image taken on one day may lead the probe incorrectly on a different day when treatment is administered.

Even probes with a laparoscope for instantaneous view of surfaces at the probe tip are deficient for some decisions. Laparoscopes are limited in their visualization capability due to their flat representation of three-dimensional (3D) anatomy and their ability to display only the most superficial surfaces. For example, blood vessels below such surfaces are evident in renderings based on CT scans and are important in decisions on where to make incisions; yet are not visible to the laparoscope.

In some past approaches, the treatments are based on one or more scans at a single time and the treatment area is expanded to treat all positions through which the target tissues may move during the treatment, e.g., expanding the treatment area beyond the target area by some amount or percentage that is expected to cover normal flexing of the soft tissue. While suitable for some applications, this approach suffers from the disadvantage that some non-target tissue is exposed to the treatment. For example, some healthy bladder and rectal tissue is subjected to radiation intended to kill cancerous prostate tissue.

In another approach, multiple scans are taken at different times and different treatments are applied for different scans. A problem with this approach is that some scans, such as CT scans, take many minutes to perform, are expensive, expose a patient to hazardous radiation, and can obstruct access to the tissue by the treatment provider, such as an interventional radiologist or a therapeutic radiation source.

In some embodiments, multiple scans are taken using technologies that are faster, cheaper, safer or less obstructive, such as ultrasound which enjoys all four advantages. However, a problem with this approach is that the scan technology does not provide the spatial resolution needed. For example, in ultrasound there is low contrast between the prostate and bladder tissue compared to CT scans, and there is more noise in the form of speckle.

Based on the foregoing, there is clear need for techniques that provide time varying determinations of internal tissue spatial arrangement in a living body that do not suffer the disadvantages of prior art approaches.

In particular, there is a need for techniques that provide time varying determinations of internal tissue spatial arrangement in a living body that provides high spatial resolution boundaries of target tissue in times short compared to changes in those boundaries that are significant for treatment.

### SUMMARY OF THE INVENTION

Techniques are provided for image-guided intervention, which do not suffer all the deficiencies of prior art approaches.

In one set of embodiments, a method for indicating current disposition of moving target tissue in a living body includes receiving high spatial resolution scan data. This data represents a scan of a living body based at least in part on a first mode of measuring the living body with high spatial resolution over a first mode measurement duration. A repeat rate for repeatedly obtaining the high spatial resolution scan data based on the first mode of measuring the living body over a treatment period of time for treating the living body is limited to be no greater than a first repeat rate. The method also includes receiving high temporal resolution scan data. This data represents a scan of the living body based at least in part on a different second mode of measuring the living body over a second mode measurement duration. Allowed repeat rates for repeatedly obtaining the high temporal resolution scan data based on the second mode of measuring the living body over the treatment period of time is greater than the first repeat rate. The method also includes determining an elastic transform that registers the high spatial resolution scan data elastically to the high temporal resolution scan data. A current spatial arrangement of a moving target tissue in the living body during the second mode measurement duration is determined based on the elastic transform. The moving target tissue changes over the treatment period among multiple spatial arrangements that are significantly different for treatment of the target tissue.

In another set of embodiments, a method for indicating disposition of moving target tissue in a living body includes receiving first scan data and second scan data. The first scan data represents a scan of a living body based at least in part on a first mode of measuring the living body with high spatial resolution at a first measurement time. The second scan data represents a scan of the living body based at least in part on a second mode of measuring the living body at a different second measurement time. A moving target tissue in the living body changes from the first measurement time to the second measurement time in a way that is significantly different for treatment of the target tissue. An elastic transform is determined, which registers the first scan data elastically to the second scan data. A particular spatial arrangement of the moving target tissue in the living body at a particular time between the first measurement time and the second measurement time is indicated by interpolating the elastic transform.

In other sets of embodiments, an apparatus or a computer-readable medium implements one or more steps of the above methods.

### BRIEF DESCRIPTION OF THE DRAWINGS

The present invention is illustrated by way of example, and not by way of limitation, in the figures of the accompanying drawings and in which like reference numerals refer to similar elements and in which:

FIG. 1 is a block diagram that illustrates an imaging system for determining spatial arrangement of moving target tissue, according to an embodiment;

FIG. 2A is a block diagram that illustrates scan elements in a 2D scan;

FIG. 2B is a block diagram that illustrates scan elements in a 3D scan;

FIG. 2C is a block diagram that illustrates different scan elements in a 3D scan;

FIG. 3A, FIG. 3B, FIG. 3C and FIG. 3D are block diagrams that illustrate transformation vectors determined during non-rigid registration, according to an embodiment;

FIG. 4 is a block diagram that illustrates new parameters for constraining an implementation of a Chain Mail procedure for non-rigid registration of two scans, according to an embodiment;

FIG. 5A, FIG. 5B, FIG. 5C are diagrams that illustrate application of the new parameters of FIG. 9, according to an embodiment;

FIG. 6A and FIG. 6B are diagram that illustrates another new parameter for constraining an implementation of a Chain Mail procedure for non-rigid registration of two scans, according to an embodiment;

FIG. 7A and FIG. 7B are echocardiograms that illustrate measurements at two stages of a biological cycle that are interpolated in time based on transformation vectors, according to an embodiment;

FIG. 8 is a flow diagram that illustrates at a high level a method for interpolating transforms that register high resolution scan data at one time to high resolution scan data at a different time, according to an embodiment;

FIG. 9 is a flow diagram that illustrates at a high level a method for registering high resolution scan data at a fixed time to high temporal resolution scan data at a different time, according to an embodiment;

FIG. 10A is a high dose CT scan that depicts a prostate and bladder;

FIG. 10B is a processed CT scan that results from processing of the CT scan of FIG. 10A, according to an embodiment;

FIG. 10C is graph that illustrates intensity histograms for regions of FIG. 10A that represent the prostate and bladder;

FIG. 10D is graph that illustrates intensity histograms for regions of FIG. 10B that represent the prostate and bladder;

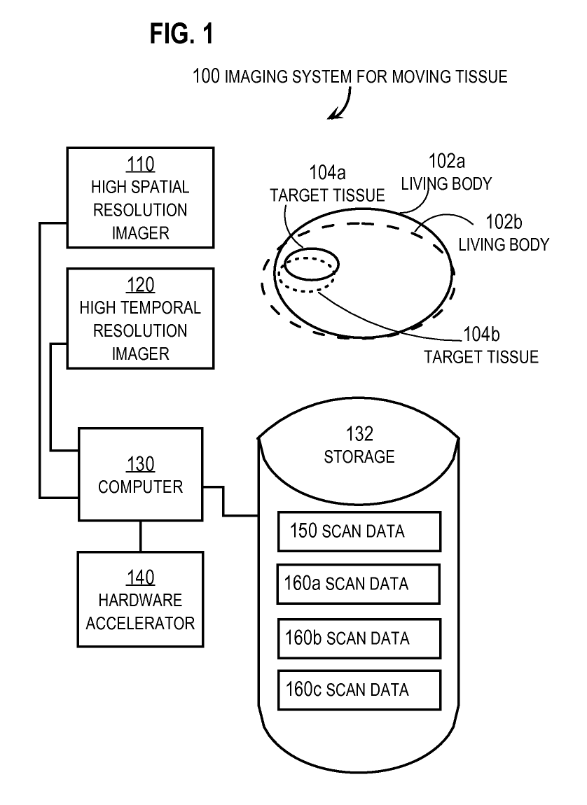

FIG. 1A is a echogram that depicts a prostate and bladder;

FIG. 1B is a processed echogram that results from processing of the echogram of FIG. 11A, according to an embodiment;

FIG. 12A is a high dose CT scan that depicts body portion including a liver;

FIG. 12B is a simulated low dose CT scan that depicts the identical body portion as depicted in FIG. 12A;

FIG. 12C is a processed low dose CT scan that results from filtering of the simulated low dose CT scan of FIG. 12B, according to an embodiment;

FIG. 13A is a high dose CT scan that depicts a body portion;

FIG. 13B is a simulated high dose CT scan that depicts the body portion depicted in FIG. 13A but deformed according to known transformation vectors;

FIG. 13C is a map that illustrates a difference between the scan of FIG. 13A and the deformed scan of FIG. 13B;

FIG. 13D is a map that illustrates a difference between the deformed scan of FIG. 13B and a transformed image formed by a non-rigid registration of the scan of FIG. 13A to the scan of FIG. 13B, according to an embodiment;

FIG. 13F is a map that illustrates a difference between the deformed scan of FIG. 13B and a transformed image formed by a non-rigid registration of the scan of FIG. 13A to a simulated low dose CT scan based on the scan of FIG. 13B, according to an embodiment; and

FIG. 14 is a block diagram that illustrates a computer system upon which an embodiment of the invention may be implemented.

### DETAILED DESCRIPTION

A method and apparatus are described for image-guided intervention for treatment of a living body. In the following description, for the purposes of explanation, numerous specific details are set forth in order to provide a thorough understanding of the present invention. It will be apparent, however, to one skilled in the art that the present invention may be practiced without these specific details. In other instances, well-known structures and devices are shown in block diagram form in order to avoid unnecessarily obscuring the present invention.

Some embodiments of the invention are described below in the context of certain applications, such as for imaging a human prostate over many days or a human lung tumor over several breathing cycles or structure below a liver surface over several hours using high dose CT scans, low dose CT scans, ultrasound scans, and PET scans. However, the invention is not limited to these contexts. In other embodiments other soft tissue temporal evolutions are determined, such as tissue around a beating heart, among others, in other human and non-human living bodies using the same or different measurement modalities, such as one or more MRI scans or laparoscope images.

1. Overview

In the embodiments described herein, a relatively high spatial resolution scan is elastically registered to another scan in order to determine soft tissue spatial arrangements at times other than the time of the high spatial resolution scan. This section provides an overview. A particular type of scan registration called Chain Mail elastic registration is described in more detail in section 2.

In a first embodiment, two or more CT scans taken at particular phases of a breathing cycle in a patient are elastically registered with each other and the registration transformation is interpolated to determine the arrangement of tissue at intervening times. This embodiment is described in more detail in section 3.

In a second embodiment, a CT scan taken at one time is registered to one or more ultrasound scans that have higher temporal resolution but lower spatial resolution. This embodiment is described in more detail in section 4. A scanning technology well suited to perform as the higher temporal resolution but lower spatial resolution is ultrasound imaging.

In a third embodiment, a full dose CT scan taken at one time is registered to one or more low dose CT scans that have higher temporal resolution but lower signal to noise. This embodiment is described in more detail in section 5.

In a fourth embodiment, a positron-emission topography (PET) scan is registered to a full dose CT scan to register lesions not readily apparent in the CT scan to features that are apparent. The high dose CT scan is then registered to one or more low dose CT scans that have higher temporal resolution but lower signal to noise. This embodiment is described in more detail in section 6.

The general problem is described herein with reference to FIG. 1. FIG. 1 is a block diagram that illustrates an imaging system for determining spatial arrangement of moving target tissue, according to an embodiment. As used herein, moving target tissue is a tissue type within a living body that changes its spatial arrangement with time in a manner that is significant for directed treatment. It is not implied that the moving target tissue necessarily does or does not undergo any net translation.

The system 100 is for determining the spatial arrangement of soft target tissue in a living body. For purposes of illustration a living body is depicted, but is not part of the system 100. In the illustrated embodiment a living body is depicted in a first spatial arrangement 102a at one time and includes a target tissue in a corresponding spatial arrangement 104a. At a different time, the same living body is in a second spatial arrangement 102b that includes the same target tissue in a different corresponding spatial arrangement 104b.

In the illustrated embodiment, system 100 includes a high spatial resolution imager 110, such as a full dose CT scanner, and a different high temporal resolution imager 120, such as a 3D ultrasound imager. In some embodiments, the high spatial resolution imager 110 is used at two or more different times and the high temporal resolution imager 120 is omitted.

In system 100, data from the imagers 110, 120 are received at a computer 130 and stored on storage device 132. Computer systems and storage devices like 130, 132, respectively, are described in more detail in a later section. Scan data 150, 160 based on data measured at imagers 110, 120 are stored on storage device 132. For example, high resolution scan data 150 is stored based on measurements from high-spatial resolution imager 110 and a set of high temporal resolution scan data 160a, 160b, 160c collected at different times (and collectively referenced hereinafter as temporal scan data 160) are also stored on storage device 132. In some embodiments, temporal scan data 160 are based on measurements by the high-spatial resolution imager 110 at different times. In some embodiments, temporal scan data 160 are based on measurements by a different imager, such as a low spatial resolution, high temporal resolution 3D ultrasound scanner.

System 140 includes a hardware accelerator 140 for speeding one or more processing steps performed on scan data 150, 160, as described in more detail below. For example, hardware accelerator 140 is implemented as an application specific integrated circuit (ASIC) as described in more detail in a later section, or a programmable gate array.

In various embodiments of the invention, temporal changes in the spatial arrangements 104a, 104b of the target tissue are determined by performing elastic registration between high resolution scan data 150 and temporal scan data 160.

Although system 100 is depicted with a particular number of imagers 110, 120, computers 130, hardware accelerators 140 and scan data 150, 160 on storage device 132 for purposes of illustration; in other embodiments more or fewer imagers 110, 120, computers 130, accelerators 140, storage devices 132 and scan data 150, 160 constitute an imaging system for determining spatial arrangement of moving tissue.

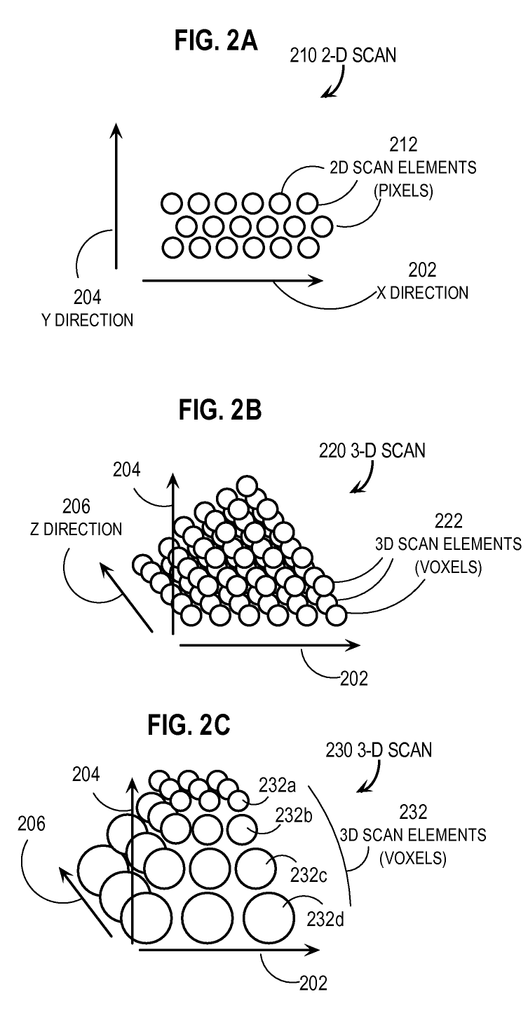

FIG. 2A is a block diagram that illustrates scan elements in a 2D scan 210, such as one slice from a CT scanner. The two dimensions of the scan 210 are represented by the x direction arrow 202 and the y direction arrow 204. The scan 210 consists of a two dimensional array of 2D scan elements (pixels) 212 each with an associated position. A value at each scan element position represents a measured or computed intensity that represents a physical property (e.g., X-ray absorption) at a corresponding position in at least a portion of the spatial arrangement 102a, 102b of the living body. Although a particular number and arrangement of equal sized circular scan elements 212 are shown for purposes of illustration, in other embodiments, more elements in the same or different arrangement with the same or different sizes and shapes are included in a 2D scan.

FIG. 2B is a block diagram that illustrates scan elements in a 3D scan 220, such as stacked multiple slices from a CT scanner. The three dimensions of the scan are represented by the x direction arrow 202, the y direction arrow 204, and the z direction arrow 206. The scan 220 consists of a three dimensional array of 3D scan elements (voxels) 222 each with an associated position. A value at each scan element position represents a measured or computed intensity that represents a physical property (e.g., X-ray absorption or acoustic reflectivity) at a corresponding position in at least a portion of the spatial arrangement 102a, 102b of the living body. Although a particular number and arrangement of equal sized spherical scan elements 222 are shown for purposes of illustration, in other embodiments, more elements in the same or different arrangement with the same or different sizes and shapes are included in a 3D scan 220.

FIG. 2C is a block diagram that illustrates different scan elements in a 3D scan 230, such as from time-gated acoustic beams in a 3D acoustic scanner. The three dimensions of the scan are represented by the x direction arrow 202, the y direction arrow 204, and the z direction arrow 206. The scan 230 consists of a three dimensional array of 3D scan elements (voxels) 232 each with an associated position. In scan 230 nine beams penetrate the volume with increasing voxel size along the beam. For example, voxels 232a, 232b, 232c, 232d represent acoustic energy returned in a corresponding four time windows that represent propagation of sound through corresponding distance segments in the living body. Although a particular number and arrangement of spherical scan elements 232 are shown for purposes of illustration, in other embodiments, more elements in the same or different arrangement with the same or different sizes and shapes are included in a 3D scan 230. For example, 3D acoustic voxels expand in size in the x-z plane formed by x-direction arrow 202 and z-direction arrow 206 but remain constant in size in the y-direction arrow 204, unlike the voxels depicted.

Certain voxels in the scan data are associated with the target tissue. The spatial arrangement of the target tissue is represented by the set of voxels that are associated with the target tissue, or by the boundary between such voxels and surrounding voxels.

In various embodiments of the invention, a first scan formed by a 2D or 3D array of scan elements is processed to identify voxels associated with the target tissue. The first scan is elastically registered to a different scan formed by a 2D or 3D array of scan elements to determine the voxels associated with the target tissue in the second scan. In some embodiments, directed treatment is administered based on the elastic registration.

Image registration is the process of aligning two or more images that represent the same object, where the images may be taken from different viewpoints or with different sensors or at different times, or some combination. A transformation that aligns two images can be classified as rigid, affine, or elastic (e.g., projective or curved). Rigid transformations include translation or rotation or both. Affine transformations add shear or scale changes or both. An elastic transformation is a special case of a non-rigid transformation that allows for local adaptivity (e.g., uses a transform that varies with position within the scan) and is typically constrained to be continuous and smooth.

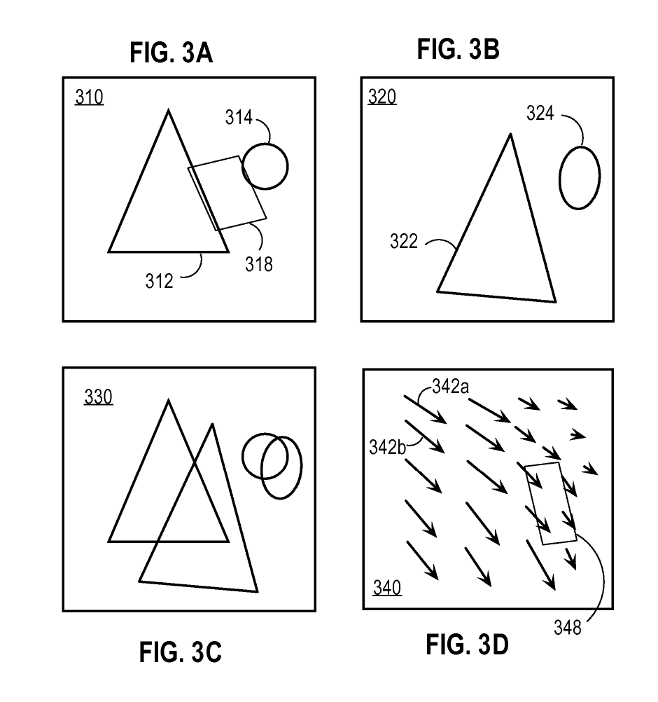

FIG. 3A, FIG. 3B, FIG. 3C and FIG. 3D are block diagrams that illustrate transformation vectors determined during an elastic registration, according to an embodiment. FIG. 3A depicts scan data 310 and target tissue boundary 318. Shapes 312 and 314 represent regions of exceptionally dark and exceptionally light voxels, respectively, in scan data 310. It is assumed, for purpose of illustration, that an expert has examined the scan data 310 and manually produced boundary 318 of the target tissue or treatment plan to indicate the edge of an organ indicted by more subtle changes in voxel intensity than indicated by shapes 312 and 314.

FIG. 3B depicts second scan data 320. Shapes 322 and 324 represent regions of exceptionally dark and exceptionally light voxels, respectively, in scan data 320. No expert examines the scan data 320. Automatic registration is performed to determine the transforms that approximately related features in scan 310 to features in scan 320, limited by the complexity and number of coefficients used to model the transformation.

FIG. 3C depicts the superposition 330 of the two scans 310 and 320. A measure of similarity is made for this overlap, and then the coefficients of the transformation are varied until the measure of similarity reaches a maximum. Any similarity measure appropriate for automatic registration of the available scan data may be used. In one illustrated embodiment, the measure of similarity is mutual information (MI) and the maximization process is as described in R. Shekhar and V. Zagrodsky, “Mutual Information-based rigid and nonrigid registration of ultrasound volumes,” IEEE Transactions in Medical Imaging, vol. 21, pp. 9-22, 2002, (hereinafter, Shekhar), the entire contents of which are hereby incorporated by reference as if fully set forth herein. The transformation that provides the maximum measure of similarity is the selected transformation.

FIG. 3D depicts an array 340 of transformation vectors. It is assumed for purposes of illustration that these transformation vectors move selected voxels of the scan 310 to corresponding voxels in scan 320 based on the selected transformation. The transformation vectors include transformation vector 342a and transformation vector 342b and others, collectively referenced herein as transformation vectors 342. Each transformation vector 342 has a tail at a position of a voxel in the original scan 310 and an arrowhead pointing to the corresponding voxel in the scan 320. The transformation provides vectors for all voxels but only a few are shown to avoid obscuring the figure.

According to some embodiments of the invention, the selected transformation is used to transform expert tissue or treatment plan boundary 318 for the reference scan data 310 to produce a transformed boundary for scan 320. It is assumed for purposes of illustration that boundary 348 in FIG. 3D is the result of transforming the boundary 318 by the selected transform vector array 340. Boundary 348 is then used to form a registered tissue or treatment plan boundary.

The non-rigid registration is performed in any manner known in the art. For example, in some embodiments, a simplified global affine transformation is applied with three translation degrees of freedom, three rotation degrees of freedom, and three compression degrees of freedom, requiring the optimization of nine parameters (the values of which are called coefficients, herein). In some embodiments, the non-rigid registration is elastic and performed using adaptive sub-volume division as described by Shekhar, cited above. In some embodiments, a 3-D Chain Mail algorithm is used to perform the elastic registration while avoiding folding artifacts. A particular adaptation of the Chain Mail algorithm to elastic registration, according to some embodiments, is described in the next section. Automatic registration is performed by defining a measure of similarity between two scans and selecting a transform that maximizes the measure of similarity. Any known measure of similarity may be used. In several illustrated embodiments, the measure of similarity is called mutual information (MI), well known in the art. In some embodiments, a root-mean-square (rms) difference is minimized to select the transform (e.g., the inverse rms difference is the measure of similarity).

In some embodiments, elastic transformations are implemented in hardware to speed the computation of the spatially dependent transforms. For example, as described in U.S. patent application Ser. No. 10/443,249 and C. R. Castro-Pareja, J. M. Jagadeesh, R. Shekhar, IEEE Transactions on Information Technology in Biomedicine, vol. 7, no. 4, pp. 426-434, 2003, the entire contents of each of which are herby incorporated by reference as if fully set forth herein, fast memory and cubic addressing are used to store and access the two scans and a mutual histogram (MH) used in the computation of MI.

2. Chain Mail Registration Improvements

The optimal deformation field values are found by maximizing an energy function which measures the similarity between the reference image and the transformed floating image. This is commonly expressed as shown in Equation 1.

$$\hat{T} = {arg max}_{T} IS ( RI ( x , y , z ) , FI ( T ( x , y , z ) ) ) , \qquad ( 1 )$$

where IS stands for image similarity, RI is the reference image, FI the floating image and T the transformation whose parameters are being optimized.

In some embodiments, a 3D Chain Mail algorithm is adapted for non-rigid registration. In a first step, a global transformation (rigid or affine) is determined. The global transformation is modeled using a transformation matrix M_{global}. In a second step, the local deformations are found. In the illustrated Chain Mail embodiment, the elastic registration algorithm uses a multi-resolution approach, where local deformations are estimated at consecutively finer grid and image resolutions. In this illustrated embodiment, local deformations are defined in the reference image space (i.e., they are applied before the global transformation). The total transformation is therefore defined by Equation 2.
\vec{v}_{FI}=M_{global}×(\vec{v}_{RI}+\vec{v}_{local}(\vec{v}_{RI})) (2)
where \vec{v}_{RI} is the location of a voxel in the reference image, \vec{v}_{FI} its corresponding location in the floating image, and \vec{v}_{local}(\vec{v}_{RI}) the value of the local deformation field at \vec{v}_{RI}=(x,y,z). The local deformation field is modeled using a linear combination of cubic B-splines placed on a regular grid of control points φ_{i, j, k}, with i<nᵢ, j<nⱼ, k<nₖ and grid spacing δₓ(t), δ_{y}(t) and δ_{z}(t):

$$\begin{matrix}{{v}^{->}}_{local} ( x , y , z ) = {\sum }_{l = - 1}^{2} {\sum }_{m = - 1}^{2} {\sum }_{n = - 1}^{2} B ( u ) B ( v ) B ( w ) {ϕ}_{i + l , j + m , k + n} , \\ where i = ⌊ x / {δ}_{x} ⌋ , j = ⌊ y / {δ}_{y} ⌋ , k = ⌊ z / {δ}_{z} ⌋ , \\ u = x / {δ}_{x} - i , v = y / {δ}_{y} - j , w = z / {δ}_{z} - k , and \text{:} & ( 3 ) \\ B ( r ) = \{ \begin{matrix}3 / 6 \cdot {r}^{3} - {r}^{2} + 4 / 6 , & r < 1 \\ - 1 / 6 \cdot {r}^{3} + {r}^{2} - 2 \cdot r + 8 / 6 & 1 \leq r < 2 \\ 0 & r \geq 2\end{matrix} & ( 4 )\end{matrix}$$

Given an initial grid spacing of δₓ(0), δ_{y}(0) and δ_{z}(0), the multi-resolution algorithm was implemented by defining δ(t)=δ(t−1)/2 for t=1 . . . nᵣₑₛₒₗᵤₜᵢₒₙₛ−1, where nᵣₑₛₒₗᵤₜᵢₒₙₛ is the total number of grid resolutions used to estimate the deformation field.

B-splines are used to model the deformation field. The illustrated embodiment differs from previous approaches using B-splines in two aspects:

1] The local deformation field is modeled in the reference image space, as opposed to in the floating image space. Modeling the local deformations in the reference image space has the advantage that it allows for an efficient implementation of the 3D Chain Mail algorithm, as shown below.

2] The control point grid is subject to internal forces that preserve the topology of the grid, thereby eliminating the occurrence of folding artifacts. These forces allow the transmission of variations in the local deformation field between neighboring control points, when necessary to preserve the grid topology.

Estimation of the deformation field at a given grid resolution is performed using an optimization algorithm to determine the optimal values for each control point. Any optimization algorithm may be used. The illustrated embodiment decomposes the global optimization problem into a set of local 3-dimensional optimization problems by optimizing one control point location at a time. At a given resolution, the illustrated embodiment first optimizes all control points in raster order, keeping track of the control points whose deformation field values are changed significantly. After the first pass, the algorithm proceeds to optimize only those control points that were significantly affected in the previous pass, and their neighbors.

The local deformation field is modeled using a 3D Chain Mail algorithm, which was introduced as a faster alternative to computationally intensive finite element methods for elastic deformation of 3D meshes. The 3D Chain Mail algorithm controls the propagation of local deformations between adjacent control points, with the goal of preserving the control point grid topology. In the illustrated embodiment, propagation of local deformations is controlled by three new parameters: minimum neighbor distance (dₘᵢₙ), maximum neighbor distance (dₘₐₓ) and maximum shear distance (sₘₐₓ). These new parameters act as bounds on the relative positions of adjacent control points, and can be defined either globally or locally, thereby allowing fine control over local deformations. They are defined independently for each direction (x, y and z) with a magnitude related to the grid spacing using the three positive constants δₘᵢₙ≦1, δₘₐₓ≧1 and σₘₐₓ as given by Equations 5, 6 and 7.
dₘᵢₙₓ(t)=δₘᵢₙ·δₓ(t),d_{miny}(t)=δₘᵢₙ·δ_{y}(t),d_{minz}(t)=δₘᵢₙ·δ_{z}(t), (5)
dₘₐₓₓ(t)=δₘₐₓ·δₓ(t),d_{maxy}(t)=δₘₐₓ·δ_{y}(t),d_{maxz}(t)=δₘₐₓ·δ_{z}(t), (6)
sₘₐₓₓ(t)=σₘₐₓ·δₓ(t),s_{maxy}(t)=σₘₐₓ·δ_{y}(t), and s_{maxz}(t)=σₘₐₓ·δ_{z}(t). (7)

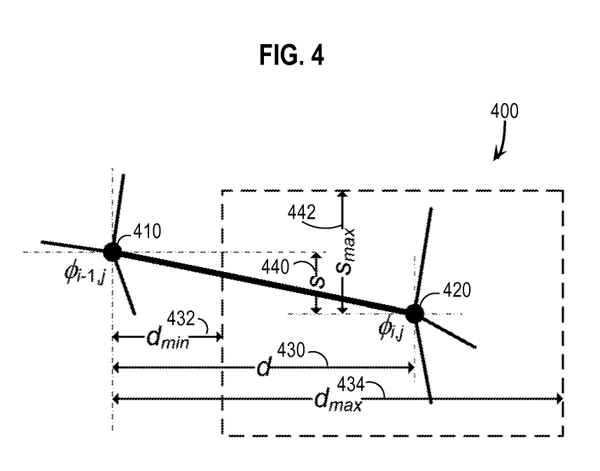

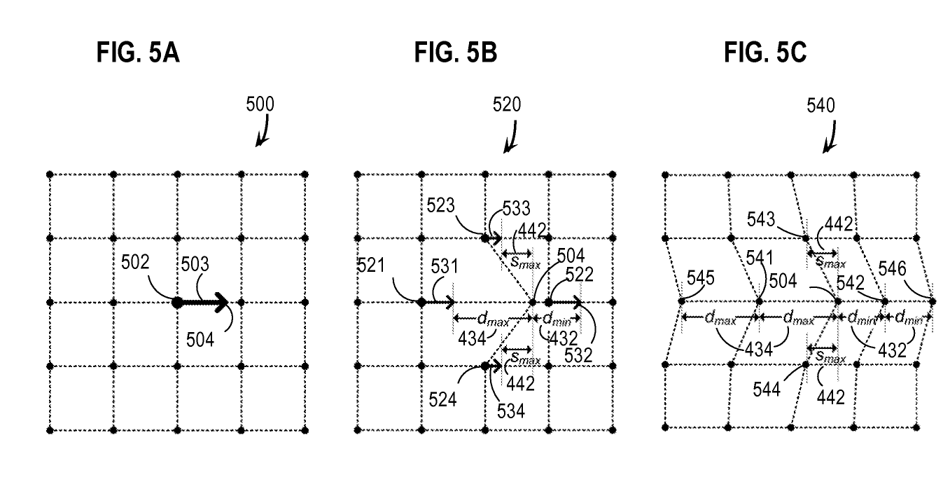

FIG. 4 is a block diagram 400 that illustrates the new parameters for constraining an implementation of a Chain Mail procedure for non-rigid registration of two scans, according to an embodiment. FIG. 4 shows how these bounds are applied in the 2D case. Given two control points 410, 420, φ_{(x,y)} and φ_{(x,y)+î}, respectively, connected in the î direction, their distance in the î direction, called the neighbor distance 430, d, must satisfy 432 dₘᵢₙ<430 d<434 dₘₐₓ, and their distance in the direction orthogonal to î, called the shear distance 440, s, must satisfy 440 s<442 sₘₐₓ. The control point 420 φ_{(x,y)+î}, can therefore be displaced to any location within the rectangle delimited with dashed lines without violating the bounds. If the bounds are violated, the position of the adjacent control point is changed to a new location that satisfies the bounds. Bound checking is then performed for the neighbors of the control point that was displaced, thus propagating the local deformation through the rest of the grid. FIG. 5A, FIG. 5B, FIG. 5C are diagrams that illustrate application of the new parameters of FIG. 4, according to an embodiment. FIG. 5A depicts an original uniform grid 500 with point 502 that is subsequently subjected to a displacement 503 to become a displaced point 504. FIG. 5B depicts the intermediate grid 520 with displaced point 504 and distances to adjacent points 521, 522, 523, 524. To preserve distances within the bounds of 432 dₘᵢₙ, 434 dₘₐₓ, and 442 Sₘₐₓ, the adjacent points are displaced by vectors 531, 532, 533 and 534, respectively. FIG. 5C depicts the adjusted grid 540 with displaced points 504, 541, 542, 543, 544 as well as more distinct adjusted points 545, 546. The adjusted grid 540 preserves distances within the bounds of 432 dₘᵢₙ, 434 dₘₐₓ, and 442 Sₘₐₓ.

To achieve the simplest implementation, the control point grid is defined in the reference image space, with control points connected along the x, y and z dimensions, such that for a given pair of connected control points in 3D, the neighbor distance corresponds to the distance in the dimension in which they are connected, and the two shear distances correspond to the distances in the remaining dimensions. Such implementation requires applying local deformations before the global transformation.

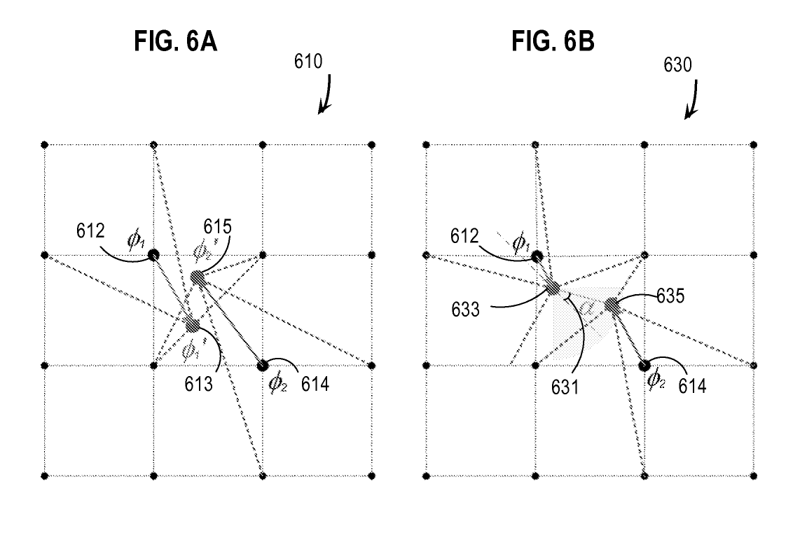

The original 3D Chain Mail algorithm does in most cases a good job of preventing folding artifacts. However, it does not guarantee a complete elimination of folding artifacts for all values of the control constants δₘᵢₙ, δₘₐₓ and σₘₐₓ. FIG. 6A and FIG. 6B are diagrams that illustrates another new parameter for constraining an implementation of a Chain Mail procedure for non-rigid registration of two scans, according to an embodiment. A special case where grid folding occurs on a control point grid that is valid according to the original 3D Chain Mail algorithm is shown in FIG. 6A. FIG. 6A depicts grid 610 with uniform grid point 612 displaced to point 613 and uniform grid point 614 displaced to point 615. The reason why this case is possible is because the 3D Chain Mail algorithm does not provide interactions between adjacent nodes in diagonal directions. There are several alternatives for preventing the occurrence of this case, described next.

1. Use σₘₐₓ<δₘᵢₙ/2. This solution is practical in most cases where the compressibility of internal structures is relatively small or where the shear component in local deformations is not significant.

2. Support propagation of local deformations between neighboring control points in diagonal directions. One way to implement such an interaction consists of limiting the direction of the vector that points from one control point to another in a diagonal direction to up to 45 degrees from the original (un-deformed) direction, e.g., 45 degrees in 2D. A 2D example is shown in FIG. 6B. In FIG. 6B the control points in grid 630 are constrained so that an angle α 631 between a segment connecting the displaced diagonal points 633 and 635 and a direction in the uniform grid is less than 45 degrees. The shaded area shows the acceptable directions of the displacement vector for point 633 after deformation.

While preventing folding artifacts ensures the mathematical validity of the transformation, it does not prevent the occurrence of local deformations with a magnitude beyond the expected range. This is especially the case in datasets with large local deformations in small parts of the image. For a given application in medical imaging, it is usually possible to determine a priori the range of reasonable shifts in the internal anatomy (i.e., the maximum distance that a voxel can travel). Such distance, called the “Maximum Voxel Displacement” (d_{MVD}) is used as an additional new parameter to restrict the magnitude of deformations at a global or at a local level. It is applied by defining a three-dimensional bounding sphere R_{MVD} that is used as an additional constraint in the optimization algorithm:
R_{MVD}={\vec{v}_{RI}εFI|∥\vec{v}_{local}(\vec{v}_{RI})∥<d_{MVD}}. (9)

Using d_{MVD} effectively reduces the search space for the local deformation value at each control point, especially at coarser grid resolutions. It allows using constrained optimization algorithms to maximize the local image similarity. It also improves algorithm robustness since unrealistic but topologically correct transformations are not considered. Knowing the maximum voxel displacement also helps in determining the starting grid resolution for the algorithm. A criteria for choosing the initial grid resolution in the illustrated embodiment is to set 2·d_{MVD}>δ_{x,y,z}(0)>d_{MVD}. In embodiments where sufficiently detailed a priori information is available, the maximum voxel displacement is used as a local value in the reference image space (e.g., d_{MVD}=d_{MVD} (\vec{v}_{RI})). Using d_{MVD} as a local value allows selecting the region of interest inside the image, where local deformations are expected to occur. It also allows selecting the regions where large deformations are expected, thus controlling the deformation magnitude at a local level. Furthermore, it allows delaying optimization of regions with small expected deformations until the condition 2·d_{MVD}(\vec{v}_{RI})>δ_{x,y,z}(t) is met, thereby reducing the total number of optimization steps required to execute the algorithm.

One aspect of importance to the Chain Mail registration is the region of influence of each control point. The region of influence of a given control point is the region over which image similarity is calculated when the local shift at the given control point is being optimized. It includes at a minimum the volume of support of the given control point. However, since shifts applied to a control point can propagate through its neighbors, the control point region of influence also includes their support volumes in the illustrated embodiment. Theoretically, a situation could arise when a shift applied to a given control point propagates through the whole grid, thereby making calculation of image similarity over the whole image necessary.

Since d_{MVD} places a bound to local shifts, it also places a bound to the number of neighboring control points that can be affected by a given control point using the 3D Chain Mail algorithm. Before optimizing each control point, the illustrated embodiment of the method calculates its corresponding region of influence by determining the set of neighboring control points that would be affected for a given range of local shifts. The first time the local deformations for a given control point are estimated, it is assumed that the range of possible shifts is ±d_{MVD} in each direction. Such an assumption is always valid. At later passes and grid resolutions it is excessively rigorous for regions that exhibited small local deformation in previous estimations. Therefore, a more computationally efficient way to determine the region of influence at a control point where an estimate of the local shift has already been calculated is to estimate the range of possible shifts to be a fraction of the current local deformation. In the illustrated embodiment, it is assumed that the local deformation does not change by more than 50% when optimizing the local shift at a control point where a local deformation estimate is already present. Such an assumption allows a dramatic reduction in the size of the region of influence of control points with small local deformations, especially at finer grid resolutions.

Many elastic registration algorithms presented in the literature include a method to determine which control point locations should be optimized and which ones should be left unaltered. Identifying active and passive control points is an efficient way to improve the performance of the algorithm, by reducing the number of degrees of freedom in the transformation and therefore the number of image similarity calculations needed to converge. Any method may be used to identify active regions.

The elastic registration of the illustrated embodiment thus includes the following steps:
1. Performing an affine registration (calculate M_{global}).
2. Creating a pyramid of images at the desired number of resolutions.
3. Creating control point grid at starting resolution (t=0).
4. For a given number of passes:
   a. Determining active control points by identifying active regions.
   b. For each active control point:
      i. Optimizing the value of the local deformation field at the control point location, subject to constraints dictated by R_{MVD}, by maximizing image similarity over either the whole volume of support of the control point.
      ii. If the local deformation field value after optimization differs significantly from the value before optimization, marking the control points whose positions have been altered, as well as all their corresponding neighbors, as active for the next iteration.
5. If all grid resolutions have been optimized, exiting.
6. Refining grid (i.e., calculating grid for t=t+1) and select the images at the next resolution level in the pyramid. Going to step 4 until exiting.

The number of passes determines how many times each control point is optimized at a given grid resolution. In general, the improvement in registration accuracy resulting from running each additional pass is lower with respect to the improvement resulting from the previous pass. In an example embodiment, the improvement in image similarity beyond the third pass was very limited. Hence, in these embodiments only three passes per grid resolution were used.

3. Interpolation in Time

In some embodiments that involve cyclically moving target tissue, as in a lung tumor and heart features, the movement is determined by using elastic registration to interpolate between scans made at two or more stages of the cycle.

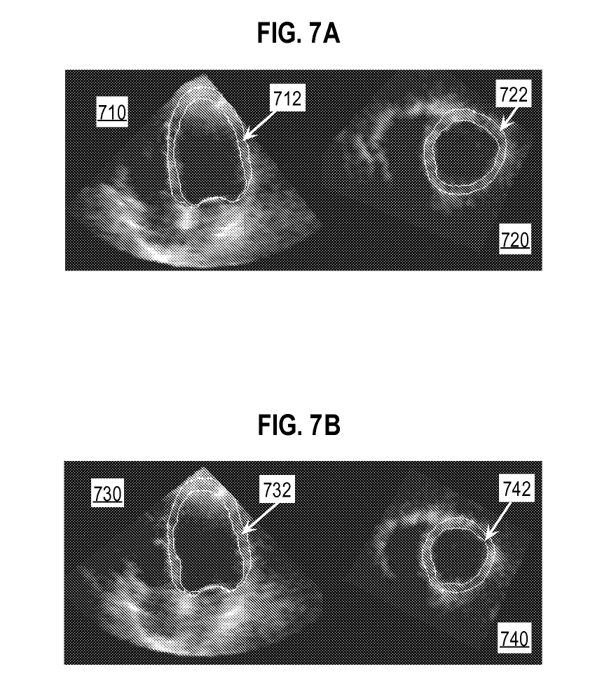

For a pictorial example of a biological cycle consider FIG. 7A and FIG. 7B. FIG. 7A and FIG. 7B are echocardiograms that illustrate measurements at two stages of a biological cardiac cycle that are interpolated in time based on transformation vectors, according to an embodiment. Scan 710 is a vertical slice that shows the myocardium 712 (the heart wall) for a left ventricle at the end of expansion (end-diastole) phase of the heart cycle. Scan 720 is a horizontal slice that shows the myocardium 722 for a left ventricle at the end-diastole phase. Scan 730 is a vertical slice that shows the myocardium 732 for a left ventricle at the end of contraction (systole) phase of the heart cycle. Scan 740 is a horizontal slice that shows the myocardium 722 for a left ventricle at the end-systole phase. In the following, different phases of the breathing cycle are considered.

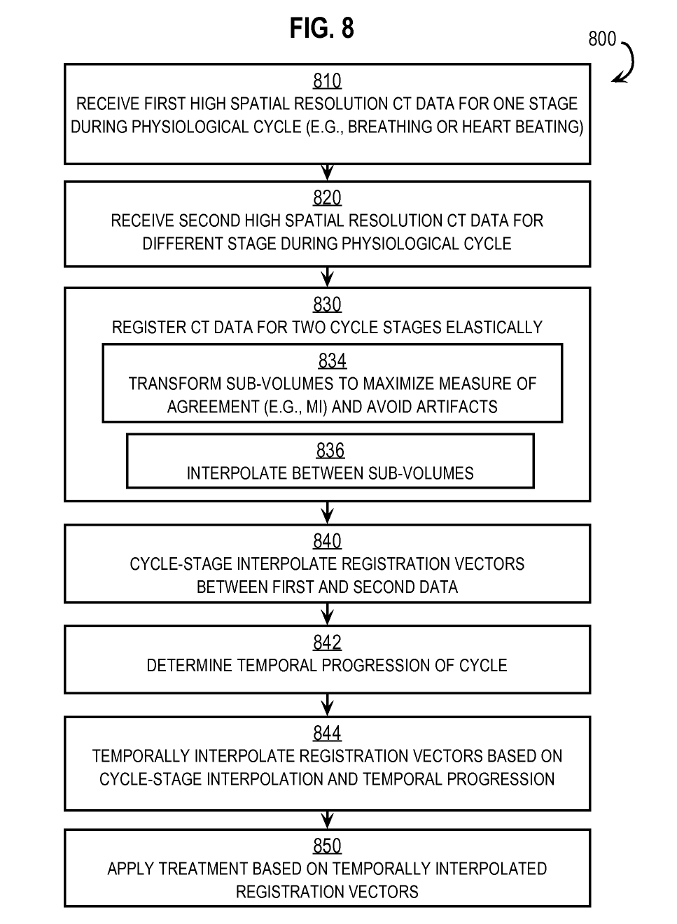

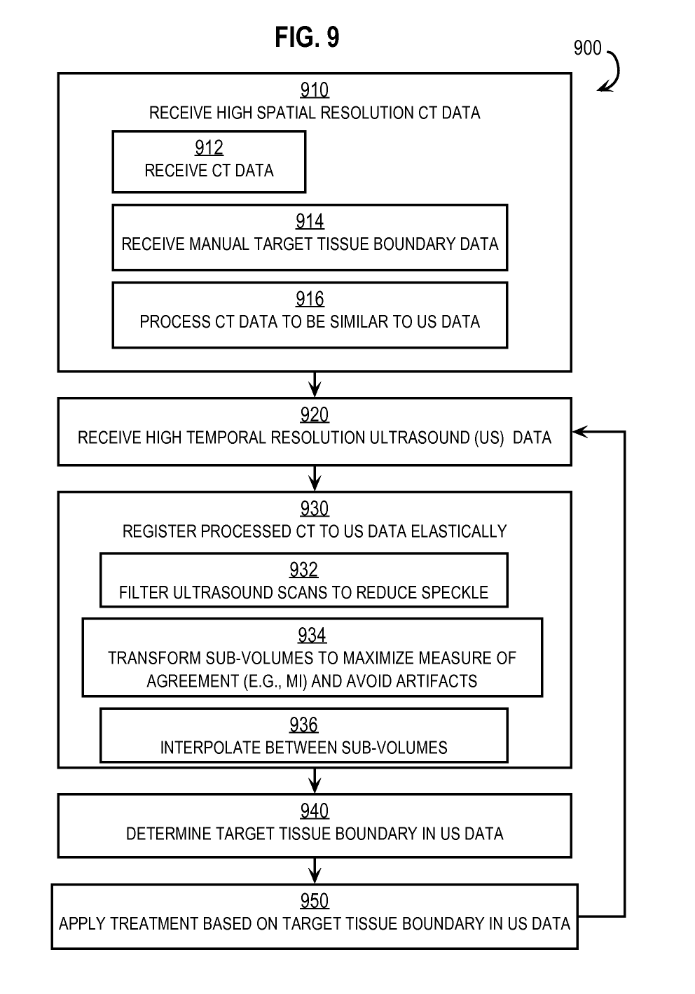

FIG. 8 is a flow diagram that illustrates at a high level a method for interpolating registration of a high resolution scan data at one time to a high resolution scan data at a different time, according to an embodiment. Although steps are shown in FIG. 8, and subsequent flow diagram FIG. 9, in a particular order for purposes of illustration, in other embodiments the steps may be performed in a different order or overlapping in time or one or more steps may be omitted and others added, or some combination of changes may occur.

In step 810, first scan data is received for one time during a physiological cycle of a living body. We use the term physiological cycle to indicate any repeating process that changes the position or shape of tissue in a living body. Examples of such cycles include the breathing cycle and the heart pumping cycle in mammals. For example, high spatial resolution CT data is received for a particular stage of a human breathing cycle, such as a complete exhale. Such images are often constructed by repeated measurements at each cycle stage, e.g., by having a patient hold a complete exhale for several seconds, while a portion of the needed CT measurements are made, allowing the patient to breath, and then again holding a complete exhale while performing an additional portion of the needed CT measurements.

Any method may be used to receive this scan data. For example, in various embodiments the scan data is received directly from a measuring device, such as imager 110, or retrieved from storage in a file or database on or connected to the computer 130 or remotely on a node of a network, either unsolicited or in response to a request for the data.

In step 820, second scan data is received for a different stage during the physiological cycle. For example, another high spatial resolution CT data is received for a particular stage of a human breathing cycle, such as a partial or complete inhale.

In step 830, an elastic transform is determined to move scan elements from the first scan to positions of associated scan elements in the second scan data. In general, the elastic transform is an array of mathematical operations associated with each scan element in the first scan. Any method may be used to determine the elastic transform. Often the elastic transform is expressed as a correction on top of a global affine transform applied to the data of the two scans.

In one illustrated embodiment, step 830 includes steps 834, 836. In step 834, the first and second scan data are broken into sub-scans (called sub-volumes even though in some embodiments one or the other is a 2D scan) and different transforms are computed for each sub-volume to maximize the measure of similarity or agreement (e.g., MI or inverse rms difference). A transform is typically represented as a vector of values for parameters that define the elements of the transform, such as 3 translation values, 3 rotation values, a shear value, and a scaling value. A different vector is allowed for each sub-volume. The sub-volumes are then further divided and incremental transforms are computed to maximize the similarity of the sub-volumes. The process continues until a minimum sub-volume size is reached. Typically the smallest sub-volume includes enough scan elements for statistically meaningful measures of similarity (e.g., MI). In the illustrated embodiment, step 834 includes avoiding folding artifacts in the transformation by applying the modified 3D Chain Mail algorithm, described above.

In step 836, the transforms associated with the smallest sub-volumes are interpolated in space to a finer scale, even, in some embodiments, down to the scale of individual scan elements of the first scan.

In step 840, the transformation vectors that register the first scan to the second scan are interpolated to fractions of the full transform vectors to represent fractional stages in the cycle between the stage represented by the first scan and the stage represented by the second scan. For convenience, this is called the cycle stage interpolation. For example, the transforms for three evenly spaced intervening stages between complete exhale and partial inhale are computing by taking one quarter, one half, and three quarters of the transform values between the first scan and the second scan at each scan element location of the first scan. In other embodiments, non-linear interpolations are performed between measured cycle stages, such as using cubic spline interpolation, well known in the art.

The steps 820, 830, 840 can be repeated to perform cycle stage interpolation between other stages of the cycle. For example the process can be repeated from partial inhale and full inhale, and from full inhale to partial exhale, and from partial exhale to complete exhale. In the illustrated embodiment, three measured stages are end inhale, mid-exhale and end-exhale. Three stages are linearly interpolated between end inhale and mid exhale and three more stages are linearly interpolated between mid-exhale and end-exhale. The one measured and six interpolated exhale stages so determined are assumed to apply in reverse for the inhale stages between end-exhale and end-inhale in the illustrated embodiment.

In step 842, the temporal evolution of the cycle is determined. For example, the breathing cycle stages change more quickly near mid-exhale than at end-inhale and end-exhale. In the illustrated embodiment, the time spent in each stage is determined based on a published diaphragm-motion waveform. In some embodiments, step 842 is omitted and the cycle stage interpolated transforms are assumed to be evenly separated in time.

In step 844, the current distribution of the target tissue is obtained by interpolating the cycle stage interpolation determined in step 840 to the current time based on the temporal progression determined in step 842; and applying the resulting interpolated transforms to the scan elements (e.g., voxels) in the first scan associated with the target tissue. The result is the location of those scan elements at the current time. In some embodiments, step 844 involves simply selecting the stage or previously computed interpolation that represents the portion of the cycle closest to the current time.

In step 850, treatment is applied based on the location of those scan elements associated with the target tissue at the current time. For example, radiation is focused at the current time on the locations in the patient that correspond to the voxels of the moving target tissue at the current time. In an illustrated embodiment, a time varying radiation dose is applied that considers the time varying spatial arrangement of the target tissue at the different stages of the breathing cycle. In the illustrated embodiment, the effects of hysterisis are ignored. In other embodiments, the effects of hysterisis are accounted for by making one or more additional CT measurements between the end exhale and end inhale phases.

The method 800 improves the efficacy or safety of a directed treatment. For example, in the illustrated embodiment, the method increases the radiation dose delivered to the lung tumor and decreases the dose delivered to healthy tissue nearby. A stronger, more effective radiation dose can be applied because less healthy tissue is exposed.

For example, weighted dose distributions were registered to the end-exhale CT data using the image transformation fields previously obtained in the registration of the CT images. The illustrated embodiment was applied to CT images obtained from a right lung tumor case. An intensity-modulated radiation therapy (IMRT) treatment plan with 5 beams was designed and the tumor prescription was 66 Gy delivered in 33 fractions with appropriate dose-volume constraints on the left and right lungs. The results were compared for the treatment plans calculated on the i) end-exhale CT images, ii) end-inhale CT images, iii) mid-exhale CT images and iv) dose registered to the end-exhale CT images using elastic registration. The mass of healthy lung receiving 20 Gy was 24.0%, 22.7%, 21.1% and 22.9% using the end-exhale, mid-exhale, end-inhale and registered data sets, respectively. The volume of the tumor receiving 100% and 90% of the prescription dose was unchanged. These results show that ignoring the effects of respiration-induced tumor motion during treatment plan evaluation can lead to incorrect estimates in dose-mass histograms for the lungs.

4. Real Time Registration with Ultrasound

In some embodiments that involve non-cyclic movement, like spatial changes to the position of the prostate, the method 800 is not appropriate. Real-time information on the spatial arrangement of the moving target tissue is desired. FIG. 9 is a flow diagram that illustrates at a high level a method 900 for registering high resolution scan data at a fixed time to high temporal resolution scan data at a different time, according to an embodiment.

In step 910, first scan data is received for one time. For example, high spatial resolution, high dose CT data is received for pre-treatment planning. Any method may be used to receive this scan data, as described above for step 810.

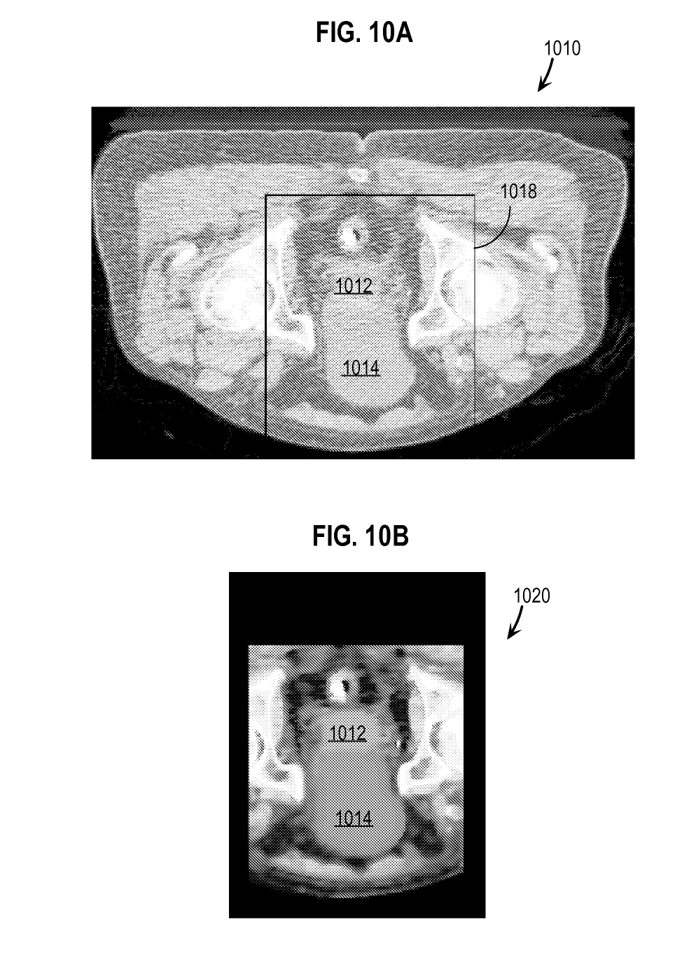

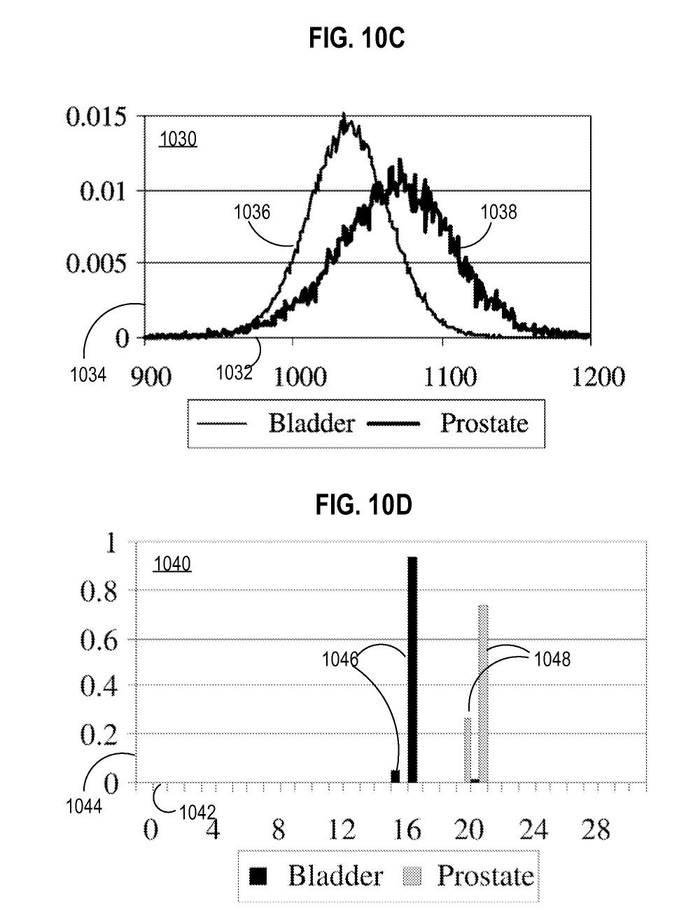

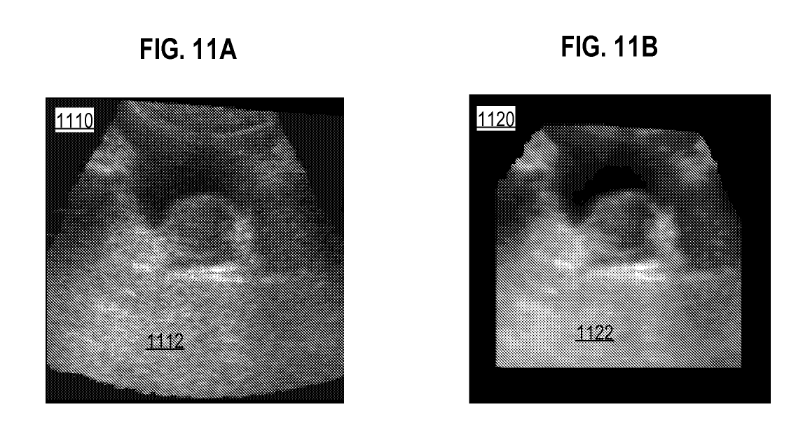

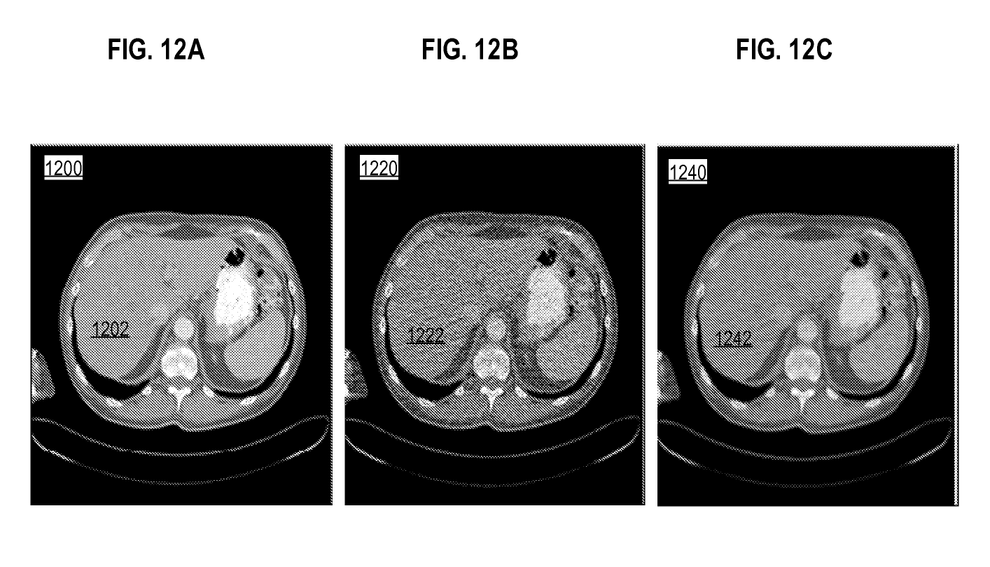

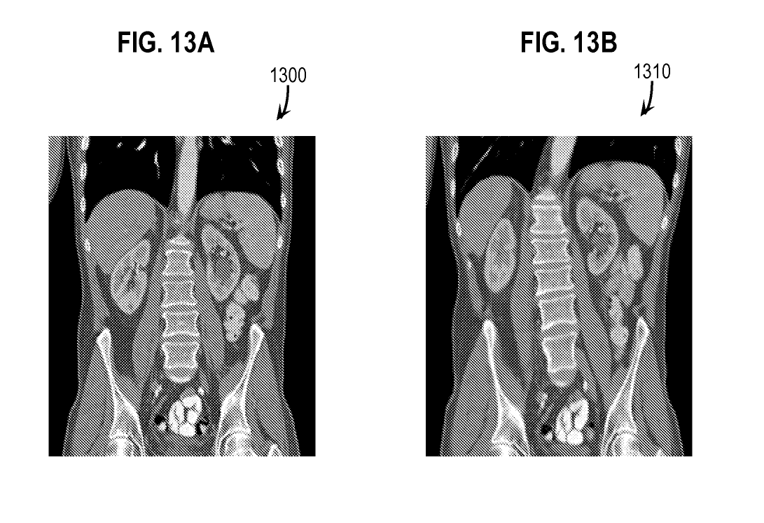

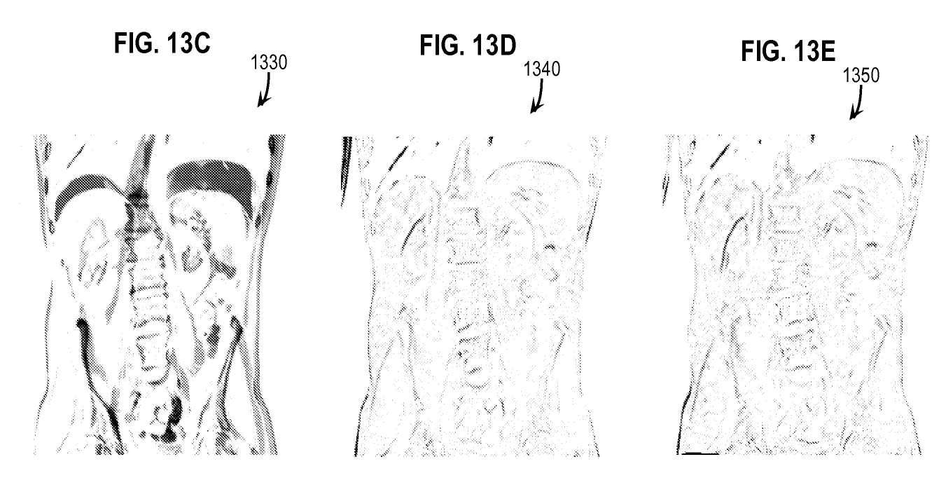

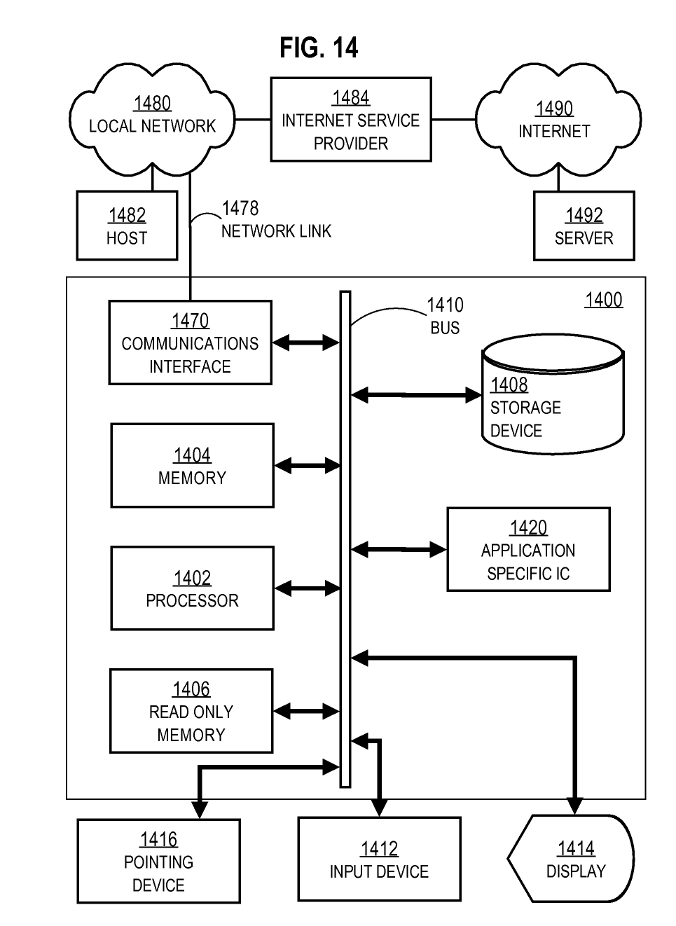

FIG. 10A is an example high dose CT scan 1010 that depicts a prostate 1012 and bladder 1014. Also depicted in FIG. 10A is an area of interest 1018.

In some embodiments, step 910 includes steps for pre-processing measured scan data to produce first scan data better suited for registering with scans from a different scanner with high temporal resolution, such as an ultrasound scanner. In the illustrated embodiment using ultrasound scans, step 910 includes steps 912, 914, 916.

In step 912, unprocessed high spatial resolution scan data is received. For example, measured CT scan data, like scan 1010, is received from imager 110. In step 914, boundary data is received that indicates a boundary between target tissue and other tissue in a scan based on the high spatial resolution data. In the illustrated embodiment, the boundary data is manually input by a human expert. For example data is received which indicates area of interest boundary 1018. In other embodiments, the boundary is determined automatically by segmenting the high spatial resolution scan data, using any scan segmentation method known in the art. In some embodiments, step 912 is omitted. For example, in some embodiments, low dose CT scan often gives good boundaries and a boundary based on the high resolution full dose CT scan is not needed.

In step 916, the high spatial resolution data is further processed to improve similarity with high temporal resolution data. For example, in an illustrated embodiment, the high spatial resolution CT scan data is processed by averaging scan element intensities within a boundary and replacing those scan element intensities with the average value. Also, in the illustrated embodiment of step 916, the scans were smoothed using Whitaker and Pfizer's anisotropic diffusion filtering algorithm with each 2D CT slice. Also, in the illustrated embodiment of step 916, the processed CT scan is then cropped to an area of interest to eliminate features that do not appear in the high temporal resolution ultrasound data. FIG. 10B is a processed CT scan 1020 that results from processing of the CT scan 1010 of FIG. 10A, according to the illustrated embodiment of step 916. As can be seen in FIG. 10B, the data has been cropped to the area of interest 1018, and the area associated with the prostate 1012 has been replaced by one average value while the area associated with bladder 1014 has been replaced by a different average value. FIG. 10C is graph 1030 that illustrates intensity histograms for regions of FIG. 10A that represent the prostate 1012 and bladder 1014 before replacement with average values. The horizontal axis 1032 represents intensity and the vertical axis 1034 represents the fraction of all pixels in the area with that intensity. The curve 1036 shows the distribution of intensities in the area associated with bladder 1014. The curve 1038 shows the distribution of intensities in the area associated with prostate 1012. FIG. 10D is graph 1040 that illustrates intensity histograms for regions of FIG. 10A that represent the prostate 1012 and bladder 1014 after replacement with average values. The horizontal axis 1032 represents intensity in terms of 32 intensity bins used to span the area of interest 1018; and the vertical axis 1034 represents the fraction of all pixels in the area with a given intensity bin. The histogram 1046 shows value 16 is associated with bladder 1014 after averaging, as plotted in FIG. 10B. The histogram 1048 shows the value 21 is associated with prostate 1012 after averaging, as plotted in FIG. 10B.

In step 920, second scan data is received for a different time using high temporal resolution data. For example, 3D ultrasound scan data is received to characterize the moving target tissue at the current time. In some embodiments, multiple 2D slices of low-dose CT scan data is received to characterize the moving target tissue at the current time, as described in the next section.

In step 930, an elastic transform is determined to move scan elements from the first scan to positions of associated scan elements in the second scan data. In the illustrated embodiment step 930 includes steps 932, 934, 936.

In step 932 the ultrasound scan data is preprocessed by filtering to reduce speckle. FIG. 11A is an echogram (ultrasound scan) 110 that depicts a prostate and bladder. The echogram 1110 is subject to speckle, as exemplified by the variable intensity shown in the relatively homogeneous area 1112. During step 932 in the illustrated embodiment, a region of interest in each ultrasound image is determined by masking out background voxels, as well as the voxels in the near and far fields. 3D anisotropic diffusion filtering is performed to reduce the speckle noise in the 3D ultrasound images. Real-time anisotropic diffusion filtering of 3D images is performed in some embodiments using the system presented in Castro-Pareja C R, Dandekar O, Shekhar R, “FPGA-based real-time anisotropic diffusion filtering of 3D ultrasound images,” Proc. SPIE, 2005, 5671, pp 123-131, the entire contents of which are hereby incorporated by reference as if fully set forth herein. The filtered 3D ultrasound images are binned to 32 intensity levels using a square quantization error minimization algorithm. FIG. 11B is a processed echogram 1120 that results from processing of the echogram 1110 of FIG. 11A, according to the illustrated embodiment of step 932. As can be seen in echogram 1120, the region of interest has been cropped and speckle in area 1122 is much reduced compared to area 1112 in echogram 1110. In the illustrated embodiment of the next section, step 932 is replaced with a step to smooth and filter the low-dose CT scans. In both embodiments, filter is performed using Whitaker and Pfizer's anisotropic diffusion filtering algorithm, as described in A. Dorati, C. Lamberti, A. Sarti, P. Baraldi, and R. Pini, “Pre-processing for 3D echocardiography,” Computers in Cardiology 1995: IGEA, Modena, Italy, 565-568, the entire contents of which are hereby incorporated by reference as if fully set forth herein.

In step 934, the first and second scan data are broken into successively finer sub-volumes as described above for step 834. In the illustrated embodiment, step 934 includes avoiding folding artifacts in the transformation by applying the Chain Mail algorithm, described above. In other embodiments, such as described in the next section, the Chain Mail algorithm is not applied.

In step 936, the transforms associated with the smallest sub-volumes are interpolated in space to a finer scale, even, in some embodiments, down to the scale of individual scan elements in the first scan, as described above for step 836.

In step 940, the transformation vectors that register the first scan to the second scan are used to transform the boundary of the moving target tissue or treatment plan to the time of the second scan data. Thus, the present location of the boundary of the target tissue is determined. In some embodiments, such as described in the next section, the boundary is evident in the transformed low-dose image and the boundary is determined in that way.

Three different registration algorithms were tested in various embodiments with different preprocessing settings. In all cases the starting point for the registration was the transformation derived from the relative locations of the 3D ultrasound probe and the CT scanner, which were measured using an optical tracking system. The tested registration methods were 1] rigid registration with translations only; 2] rigid registration with rotations and translations; and 3] elastic registration. Translations and rotations were constrained to 15 millimeters (mm, 1 mm=10⁻³ meters) and 5 degrees from the starting position, respectively.

Registration was performed using an image similarity maximization algorithm based on mutual information (MI). Elastic registration was performed with a deformation field modeled using a 3D grid of B-Splines. The grid divided the CT image into 8×8×8 subvolumes. The elastic registration algorithm did not perform an initial rigid registration step. The deformation field compressibility and rigidity were controlled using the 3D Chain Mail method described above. Tissue compressibility was limited to 25%. Internal shear was limited to 15%.

Using the transformation given by the optical tracker as initial parameters, elastic registration was able to successfully localize the prostate in up to 93.3% of the cases. The best results were achieved when preprocessing both planning CT and daily 3D ultrasound images. Interestingly, performing a rigid registration with translation and rotation components did not improve the success rate of the algorithm when compared to the translation-only case

The steps 920, 930, 940 are repeated in some embodiments to advance the target tissue boundary to the time of the next high temporal resolution scan data. For example the process can be repeated to determine the moving target tissue boundary at the time of the next ultrasound scan. The illustrated elastic registration processes iterate from an initial transformation to find the best elastic transform. The closer the initial transformation is to the solution, the faster the convergence to the best elastic transform solution. Thus, repeating steps 920, 930, 940 starting with the transform for the last time can be expected to more rapidly register the processed CT scan to the next ultrasound scan. The approach of incrementally registering successive high temporal resolution scans speeds the registration and makes the method even more suitable for real-time and near-real-time procedures, such as invasive radiology.

In step 950, treatment is applied based on the location of those voxels associated with the target tissue or treatment boundary at the current time. For example, radiation is focused at the current time on the locations in the patient that correspond to the voxels of the moving target tissue at the current time. In the illustrated embodiment, the radiation planned for the CT spatial arrangement of the prostate is applied to the spatial arrangement of the prostate determined on a particular day based on a daily ultrasound scan. In some embodiments, step 950 includes volume rendering of the registered high resolution scan to aid a physician in applying treatment. In some embodiments, step 950 includes automated control of a treatment delivery system, such as a multi-leaf-collimator radiation source.

Achieving good results in automatic registration of CT and 3D ultrasound datasets involves a set of preprocessing steps that maximize the similarity between the CT and the 3D ultrasound images. A significant problem in prostate datasets is that the contrast between the prostate and the bladder in CT images is not sufficient to achieve acceptable results in mutual information-based registration. In the illustrated embodiment, this problem is solved by exploiting the manually traced contours of the prostate and the bladder, which are used in treatment planning, as guides to increase the contrast between both organs in the CT images. Another problem is different fields of view in CT and 3D ultrasound images. Since CT has a larger field of view than 3D ultrasound, the CT was cropped to roughly match the field of view of 3D ultrasound, thus preventing the presence of spurious maxima of the image similarity function in places that do not correspond to the actual solution. It is also found that cropping the boundaries of the 3D ultrasound image, which tend to suffer from artifacts such as the presence of bright, noisy patterns in the near field and dark regions in the far field, improves performance. Another step that is shown to be beneficial to the overall registration accuracy is preprocessing the 3D ultrasound image using anisotropic diffusion to reduce speckle noise. Following these preprocessing steps, we were able to perform automatic, mutual information-based registration of the CT and 3D ultrasound datasets, obtaining localization accuracy comparable to that achieved by human experts.

The method 900 is especially effective when the time to perform steps 920, 930, 940 is on the same order as the time that changes in the spatial arrangement of the target tissue occur that are significant for treatment. Thus method 900 is easily performed with software on a general purpose computer for daily prostate updates. In some embodiments using ultrasound, the repeat rate for the high temporal resolution scan data is several per second. Sub-second elastic registration can then give essentially continuous spatial distributions of moving tissue. For breathing and heart beat time scales, it is anticipated that hardware implementations of the registration process performed in step 930 working with 3D ultrasound imagers can provide the desired speed.

3D real time visualization of the anatomy using high dose CT and 3D ultrasound is also reported to greatly improve the accuracy of needle insertion and placement during liver radiofrequency ablation (RFA). In these embodiments, the elastic image registration in step 930 was performed by a Rueckert algorithm which incorporates free-form deformation based on B-splines over a uniform 3D mesh of control points. Normalized mutual information (NMI) was used as a voxel-based similarity measurement. A study was conducted on an interventional 3D abdominal multi-modality phantom (CIRS Model 057). 3D ultrasound images were acquired during step 920 using Philips SONOS 7500 scanner with an ×4 2D-matrix array probe, capable of real-time volumetric acquisition. Ultrasound images were filtered by anisotropic diffusion filtering in step 932. The planning CT scan was registered with each ultrasound frame in the volumetric sequence during step 930. The transformed CT images, whose resolution matched the resolution of the original CT scan, were continuously volume rendered in step 950 to provide virtual real time CT image. The accuracy of image registration was evaluated by manually identifying 10 landmarks in CT and ultrasound images. Root mean square (RMS) distance between homologous landmarks was computed before and after registration. Visual inspection indicated improved spatial matching of landmarks and structures following image registration. The RMS distance improved from initial 10.4 mm to 3.2 mm after registration. The variability in landmark identification by different experts did not show statistically significant difference.

5. Real Time Registration with Low Dose CT

The method 900 may be applied in any embodiment in which the high temporal resolution scan step 920 and registration step 930 can be repeated in a repeat time shorter than the repeat time involved in receiving a new scan of the high spatial resolution data. Individual ultrasound scans can be obtained much faster than CT scans because the measurement duration is shorter, and thus several ultrasound scans can be taken in the time of one CT scan. Individual low-dose scans can be obtained much more often than full dose CT scans not because the measurement duration is shorter, indeed, the measurement durations are about the same; rather, because the living body can safely be exposed to more low-dose CT scans in any time interval than to the full-dose or near-full dose CT scans of the high spatial resolution scan

FIG. 12A is a high dose CT scan 1200 that depicts a body portion including a liver as a large relatively homogeneous area 1202. A high dose scan as used herein involves the standard-dose CT images produced by 200 milliAmpere (mA)-seconds (1 mA=10⁻³ Amperes, 1 Ampere=1 Coulomb per second, 1 Coulomb≈6.24150948×10¹⁸ electrons) of 120 kiloVolt (KV) electrons (1 KV=10³ Volts). FIG. 12B is a simulated low dose CT scan that depicts the identical body portion as depicted in FIG. 12A produced by 10 mA seconds (mAs), i.e., one twentieth the standard dose.

Low dose images, such as scan 1220, were generated from a standard dose abdominal scan using syngo-based Somaris/5 simulator from SIEMENS. This simulator models the noise and attenuation effects at lower radiation doses and can generate low-dose equivalent images from an input standard-dose image. The performance and accuracy of this simulator has been previously reported. This approach ensures that scans at all radiation doses represent exactly the same anatomy.

The low-dose scans show the exact same anatomy as in the standard dose scan but are characterized by a high quantum noise. This noise is indicated by the texture in area 1222 that corresponds to area 1202 in high dose CT scan 1200. Using low-dose images, such as scan 1220, as is, for image registration might cause the dispersion of the mutual histogram (due to noise, in an otherwise uniform structure) leading to poor image registration. Anisotropic diffusion filtering has been shown to be an effective processing step prior to advanced image processing for ultrasound. FIG. 12C is a processed low dose CT scan 1240 that results from anisotropic diffusion filtering of the simulated low dose CT scan 1220 of FIG. 12B, according to an embodiment. Scan 1240 shows improvement in the visual quality after processing of the low-dose scan 1220.

It is here demonstrated using simulations that a low dose scan, such as 1220, can be used to register a high dose CT scan to the time of the low dose measurement. The validation strategy tests how well the method 900 recovers a user-introduced, known elastic deformation. This strategy is implemented in three main steps: 1) introduce the same known deformation in high dose and low-dose CT images 2) elastically register the (preoperative) standard-dose image with the (intraoperative) low-dose images 3) Compare the transformation field obtained after image registration with the original, user-introduced deformation field to calculate the registration accuracy at various doses.

FIG. 13A is a high dose CT scan 1300 that depicts an abdominal section of a body. FIG. 13B is a simulated high dose CT scan 1300 that depicts the abdominal section depicted in FIG. 13A—but deformed according to known transformation vectors. This deformed scan 1310 typifies the deformations observed in a body over time. FIG. 13C is a map 1330 that illustrates a difference between the scan 1300 of FIG. 13A and the deformed scan 1310 of FIG. 13B. The greater the difference in voxel values, the darker the pixel in map 1330. FIG. 13D is a map 1340 that illustrates a difference between the deformed scan 1310 of FIG. 13B and a transformed image formed by a non-rigid registration of the scan 1300 of FIG. 13A to the scan 1310 of FIG. 13B, according to an embodiment. FIG. 13F is a map 1350 that illustrates a difference between the deformed scan 1310 of FIG. 13B and a transformed image formed by a non-rigid registration of the scan 1300 of FIG. 13A to a simulated low dose CT scan based on the scan 1310 of FIG. 13B, according to an embodiment.

As can be seen, the difference maps 1340 and 1350 are nearly identical. This similarity indicates that the low dose scan is as effective as a high dose scan in elastically registering a planning CT scan to a real time measurement. Visually correct registration of the standard-dose image with the deformed images at various low doses (evident from the reduced features in the difference image) demonstrates the feasibility of elastic registration at low CT doses. Inter-registration errors indicate that registration results at lower doses are comparable to those obtained using a standard dose.

The process of elastic registration attempts to recover any misalignment between the reference and floating images. A perfect registration completely recovers this misalignment and yields an elastic transformation field that is identical to the deformation field representing the original misalignment. A comparison between these two fields can be used as a performance index of the registration algorithm.

In this simulation, the deformation field introduced (DFᵢ) is known at every voxel. The volume subdivision-based elastic registration algorithm generates the transformation field (RFⱼ) during step 930, which provides the transformation at every voxel in scan with dose j. The average of the magnitude of the vector differences between these two fields is reported. This average was calculated over the region of the image which contains sufficient part of the subject and hence information to yield meaningful registration. The regions of the image which contain no information (very low entropy) (e.g. black areas surrounding the subject) are masked out using a simple threshold operation.

The results show a maximum error of 11% and 9% at the doses of 10 mAs and 20 mAs, respectively. The minimum errors at these doses are 6% and 5%, respectively. As expected, the average error improves steadily with dose. Primary causes of the baseline registration error are the resolution of the images and lowest subvolume size of the registration algorithm. Further simulations with higher resolution images show that intraoperative tissue shifts can be tracked with an accuracy of 2 mm even at an x-ray tube current of 10 mAs for the high temporal resolution data received during step 920. This is equivalent to a 94% and 95% reduction in the surface and the deep tissue dose, respectively, indicating that continuous CT can provide safe and accurate surgical guidance.

Low dose CT scans can also be applied to laparoscopic procedures. Minimally invasive surgeries performed under laparoscopic guidance lead to improved patient outcomes, less scarring and significantly faster patient recovery as compared to conventional open surgeries. Rigid endoscopes (laparoscopes) are used to visualize internal anatomy and guide laparoscopic surgeries. Laparoscopes, however, are limited in their visualization capability due to their flat representation of three-dimensional (3D) anatomy and their ability to display only the most superficial surfaces. A surgeon is thus unable to see beneath visible surfaces, affecting the precision of current-generation laparoscopic surgeries. Awareness of the 3D operative field is a long-standing need of laparoscopic surgeons that laparoscopes are fundamentally limited in meeting.

According to one embodiment of method 900, continuous computed tomography (CT) of the operative field is collected in step 920 used during step 930 to produce a registered CT scan in step 940 and rendered in step 950 as a supplementary imaging tool to guide laparoscopic surgeries. 3D visualization of anatomical structures from CT data is common in diagnostic radiology. Moreover, it is possible to expose hidden structures or to see inside organs by “peeling off” outer layers by making corresponding voxels transparent. The recent emergence of 64-slice CT as well as its continuing evolution in speed and volumetric coverage makes it an ideal candidate for four-dimensional (3D space+time) intraoperative imaging. Cost and availability considerations and the ability to image across pneumoperitoneum (caused by CO2 insuffulation) also favor the use of CT scans.

In this embodiment, a standard CT image is obtained preoperatively (following pneumoperitoneum) during step 910 and the dynamic operative field is scanned using ultra low-dose CT once surgery begins during step 920. Using high-speed non-rigid 3D image registration techniques during step 930 the preoperative CT image is rapidly registered to low-dose intraoperative CT images. Registered preoperative CT images, which match the intraoperative anatomy, are then substituted for the low-dose images in step 940, are 3D rendered and presented to the surgeon during step 950.

An advantage of this approach is that, for example, hepatic vessels hidden beneath the liver surface, which are not visible to the laparoscope, are visible in the registered CT view. Visualization of critical structures, such as the vasculature, is important before making surgical dissections. Another advantage of registered volumetric CT generated view is the ability to interact and see below structures which would be opaque in traditional laparoscopic view.

Unlike 3D roadmaps created in computer-assisted neurosurgery, in these embodiments, the 3D roadmap generated in step 950 is refreshed in real time or near real time to display tissue motion and surgical manipulations along with any surgical instruments within the operative field. The use of high-speed 3D image registration also provides for dose reduction and vessel visualization. It is anticipated that these embodiments will initiate a new generation of minimally invasive surgeries relying on real-time 3D guidance. Incorporation of real-time 3D visualization and guidance is expected to allow laparoscopic surgeons to perform existing surgeries more precisely with fewer complications. Aided by improved visualization, it is also expected that many surgeries that are currently performed in an open invasive fashion can instead be performed minimally invasively thereby reducing the mortality and morbidity rates.

6. Real Time Registration of PET Scan with Low Dose CT

Performing resection of malignant liver tumors has been shown to increase the five-year survival rate of patients suffering with liver cancer. However, the majority of hepatic tumors are considered unresectable at diagnosis, due either to their anatomic location, size, or number or inadequate viable liver tissue and morbidity. Radiofrequency ablation (RFA) is emerging as a primary mode of treatment in these cases. These procedures are conventionally performed under fluoroscopic or ultrasound guidance. With the advent of multi-detector CT, many of these procedures are now being carried out under volumetric CT guidance. This volumetric CT scan provides better 3D orientation, however, some lesions, particularly small untreated masses or recurrent or residual tumors within a large treated mass are not clearly visible in CT scans. These active lesions show up clearly as regions with high uptake on a conventional emission PET scan.

In this embodiment, the instantaneous CT received during step 920 is augmented with a pre-existing PET scan received during step 910 for better identification and localization of targets during RF ablation of liver tumors. Our preliminary results indicate the registration accuracy of the order of the resolution of the PET images.

A pre-ablation CT that is used for guidance is also received during step 910 in this embodiment. The pre-ablation CT and the pre-existing PET are acquired at different times and on different scanners and hence are inherently misaligned. Non-rigid registration is performed using a mutual information (MI)-based deformable registration algorithm that utilizes volume subdivision. The PET features are then aligned with features in the pre-ablation CT during step 910, e.g., during step 916. The PET features mark the treatment plan as indicated by boundary 318 in FIG. 3A.

The non-rigid registration also provides for a fast alignment between the pre-existing PET and current low dose CT during step 930.

The accuracy of the PET-low dose CT registration is evaluated here by comparing the alignment of anatomic structures, both qualitatively and quantitatively. The qualitative assessment was performed by a clinical expert, trained in interpreting PET-CT images via visual inspection. The quantitative evaluation compares the alignment of several anatomical landmarks as predicted by the registration against a reference. Because of the lack of a gold standard for this registration, the ability of clinical experts to locate landmarks in both CT and PET is assumed as a suitable benchmark. Since this registration is targeted towards improving identification and localization of liver lesions, the ideal method to evaluate the registration accuracy quantitatively would be to evaluate the alignment of these lesions. These lesions, however, are not clearly visible on CT which precludes a meaningful quantitative comparison based on the alignment of the lesions. This problem is addressed by evaluating the registration accuracy at landmarks physically close to these lesions

This registration was tested on two clinical cases. The CT scans had dimensions and resolution of 256×256×100-150 and 1.59 mm×1.59 mm×2.5-3.0 mm respectively. The corresponding pre-existing PET scans had dimensions and resolution of 150×150×134-235 and 4.0 mm×4.0 mm×4.0 mm respectively. The average registration time for these two cases was 30 minutes, which could be further improved to approximately a minute by using accelerated hardware implementation referenced above. The results after registration and fusion show improved alignment.

For quantitative analysis, a clinical expert was asked to identify and mark well-described landmarks in both imaging modalities. The alignment between these landmarks was computed after elastic registration and compared with the position identified by the expert. The Euclidian distance between these locations, averaged for the two cases, indicate the registration accuracy of the order of the resolution of the PET images. The lowest average registration accuracy achieved (˜6.5 mm) is lower than the approximate 10-mm treatment margin interventional radiologists currently add to the visible tumor volume.

7. Computer Hardware Overview

FIG. 14 is a block diagram that illustrates a computer system 1400 upon which an embodiment of the invention may be implemented. Computer system 1400 includes a communication mechanism such as a bus 1410 for passing information between other internal and external components of the computer system 1400. Information is represented as physical signals of a measurable phenomenon, typically electric voltages, but including, in other embodiments, such phenomena as magnetic, electromagnetic, pressure, chemical, molecular atomic and quantum interactions. For example, north and south magnetic fields, or a zero and non-zero electric voltage, represent two states (0, 1) of a binary digit (bit). A sequence of binary digits constitutes digital data that is used to represent a number or code for a character. A bus 1410 includes many parallel conductors of information so that information is transferred quickly among devices coupled to the bus 1410. One or more processors 1402 for processing information are coupled with the bus 1410. A processor 1402 performs a set of operations on information. The set of operations include bringing information in from the bus 1410 and placing information on the bus 1410. The set of operations also typically include comparing two or more units of information, shifting positions of units of information, and combining two or more units of information, such as by addition or multiplication. A sequence of operations to be executed by the processor 1402 constitute computer instructions.

Computer system 1400 also includes a memory 1404 coupled to bus 1410. The memory 1404, such as a random access memory (RAM) or other dynamic storage device, stores information including computer instructions. Dynamic memory allows information stored therein to be changed by the computer system 1400. RAM allows a unit of information stored at a location called a memory address to be stored and retrieved independently of information at neighboring addresses. The memory 1404 is also used by the processor 1402 to store temporary values during execution of computer instructions. The computer system 1400 also includes a read only memory (ROM) 1406 or other static storage device coupled to the bus 1410 for storing static information, including instructions, that is not changed by the computer system 1400. Also coupled to bus 1410 is a non-volatile (persistent) storage device 1408, such as a magnetic disk or optical disk, for storing information, including instructions, that persists even when the computer system 1400 is turned off or otherwise loses power.

Information, including instructions, is provided to the bus 1410 for use by the processor from an external input device 1412, such as a keyboard containing alphanumeric keys operated by a human user, or a sensor. A sensor detects conditions in its vicinity and transforms those detections into signals compatible with the signals used to represent information in computer system 1400. Other external devices coupled to bus 1410, used primarily for interacting with humans, include a display device 1414, such as a cathode ray tube (CRT) or a liquid crystal display (LCD), for presenting images, and a pointing device 1416, such as a mouse or a trackball or cursor direction keys, for controlling a position of a small cursor image presented on the display 1414 and issuing commands associated with graphical elements presented on the display 1414.

In the illustrated embodiment, special purpose hardware, such as an application specific integrated circuit (IC) 1420, is coupled to bus 1410. The special purpose hardware is configured to perform operations not performed by processor 1402 quickly enough for special purposes. Examples of application specific ICs include graphics accelerator cards for generating images for display 1414, cryptographic boards for encrypting and decrypting messages sent over a network, speech recognition, and interfaces to special external devices, such as robotic arms and medical scanning equipment that repeatedly perform some complex sequence of operations that are more efficiently implemented in hardware.

Computer system 1400 also includes one or more instances of a communications interface 1470 coupled to bus 1410. Communication interface 1470 provides a two-way communication coupling to a variety of external devices that operate with their own processors, such as printers, scanners and external disks. In general the coupling is with a network link 1478 that is connected to a local network 1480 to which a variety of external devices with their own processors are connected. For example, communication interface 1470 may be a parallel port or a serial port or a universal serial bus (USB) port on a personal computer. In some embodiments, communications interface 1470 is an integrated services digital network (ISDN) card or a digital subscriber line (DSL) card or a telephone modem that provides an information communication connection to a corresponding type of telephone line. In some embodiments, a communication interface 1470 is a cable modem that converts signals on bus 1410 into signals for a communication connection over a coaxial cable or into optical signals for a communication connection over a fiber optic cable. As another example, communications interface 1470 may be a local area network (LAN) card to provide a data communication connection to a compatible LAN, such as Ethernet. Wireless links may also be implemented. For wireless links, the communications interface 1470 sends and receives electrical, acoustic or electromagnetic signals, including infrared and optical signals, that carry information streams, such as digital data. Such signals are examples of carrier waves.

The term computer-readable medium is used herein to refer to any medium that participates in providing information to processor 1402, including instructions for execution. Such a medium may take many forms, including, but not limited to, non-volatile media, volatile media and transmission media. Non-volatile media include, for example, optical or magnetic disks, such as storage device 1408. Volatile media include, for example, dynamic memory 1404. Transmission media include, for example, coaxial cables, copper wire, fiber optic cables, and waves that travel through space without wires or cables, such as acoustic waves and electromagnetic waves, including radio, optical and infrared waves. Signals that are transmitted over transmission media are herein called carrier waves.

Common forms of computer-readable media include, for example, a floppy disk, a flexible disk, a hard disk, a magnetic tape, or any other magnetic medium, a compact disk ROM (CD-ROM), a digital video disk (DVD) or any other optical medium, punch cards, paper tape, or any other physical medium with patterns of holes, a RAM, a programmable ROM (PROM), an erasable PROM (EPROM), a FLASH-EPROM, or any other memory chip or cartridge, a carrier wave, or any other medium from which a computer can read.

Network link 1478 typically provides information communication through one or more networks to other devices that use or process the information. For example, network link 1478 may provide a connection through local network 1480 to a host computer 1482 or to equipment 1484 operated by an Internet Service Provider (ISP). ISP equipment 1484 in turn provides data communication services through the public, world-wide packet-switching communication network of networks now commonly referred to as the Internet 1490. A computer called a server 1492 connected to the Internet provides a service in response to information received over the Internet. For example, server 1492 provides information representing video data for presentation at display 1414.

The invention is related to the use of computer system 1400 for implementing the techniques described herein. According to one embodiment of the invention, those techniques are performed by computer system 1400 in response to processor 1402 executing one or more sequences of one or more instructions contained in memory 1404. Such instructions, also called software and program code, may be read into memory 1404 from another computer-readable medium such as storage device 1408. Execution of the sequences of instructions contained in memory 1404 causes processor 1402 to perform the method steps described herein. In alternative embodiments, hardware, such as application specific integrated circuit 1420, may be used in place of or in combination with software to implement the invention. Thus, embodiments of the invention are not limited to any specific combination of hardware and software.

The signals transmitted over network link 1478 and other networks through communications interface 1470, which carry information to and from computer system 1400, are exemplary forms of carrier waves. Computer system 1400 can send and receive information, including program code, through the networks 1480, 1490 among others, through network link 1478 and communications interface 1470. In an example using the Internet 1490, a server 1492 transmits program code for a particular application, requested by a message sent from computer 1400, through Internet 1490, ISP equipment 1484, local network 1480 and communications interface 1470. The received code may be executed by processor 1402 as it is received, or may be stored in storage device 1408 or other non-volatile storage for later execution, or both. In this manner, computer system 1400 may obtain application program code in the form of a carrier wave.

Various forms of computer readable media may be involved in carrying one or more sequence of instructions or data or both to processor 1402 for execution. For example, instructions and data may initially be carried on a magnetic disk of a remote computer such as host 1482. The remote computer loads the instructions and data into its dynamic memory and sends the instructions and data over a telephone line using a modem. A modem local to the computer system 1400 receives the instructions and data on a telephone line and uses an infra-red transmitter to convert the instructions and data to an infra-red signal, a carrier wave serving as the network link 1478. An infrared detector serving as communications interface 1470 receives the instructions and data carried in the infrared signal and places information representing the instructions and data onto bus 1410. Bus 1410 carries the information to memory 1404 from which processor 1402 retrieves and executes the instructions using some of the data sent with the instructions. The instructions and data received in memory 1404 may optionally be stored on storage device 1408, either before or after execution by the processor 1402.

8. Extensions and Alternatives

In the foregoing specification, the invention has been described with reference to specific embodiments thereof. It will, however, be evident that various modifications and changes may be made thereto without departing from the broader spirit and scope of the invention. The specification and drawings are, accordingly, to be regarded in an illustrative rather than a restrictive sense.

What is claimed is:

1. A method for indicating current disposition of moving target tissue in a living body, comprising:
receiving high spatial resolution scan data representing a scan of a living body based at least in part on a first mode of measuring the living body with high spatial resolution over a first mode measurement duration, wherein a repeat rate for repeatedly obtaining the high spatial resolution scan data based on the first mode of measuring the living body over a treatment period of time for treating the living body is limited to be no greater than a first repeat rate;
receiving high temporal resolution scan data representing a scan of the living body based at least in part on a different second mode of measuring the living body over a second mode measurement duration, wherein a repeat rate for repeatedly obtaining the high temporal resolution scan data based on the second mode of measuring the living body over the treatment period of time is greater than the first repeat rate;
determining an elastic transform that registers the high spatial resolution scan data elastically to the high temporal resolution scan data; and
indicating a current spatial arrangement of a moving target tissue in the living body during the second mode measurement duration based on the elastic transform,
wherein the moving target tissue changes over the treatment period among a plurality of spatial arrangements of the moving target tissue that are significantly different for treatment of the target tissue.

2. A method as recited in claim 1, wherein the second mode of measuring the living body is three dimensional ultrasound.

3. A method as recited in claim 1, wherein the second mode of measuring the living body is two-dimensional computer-aided low dose X-ray tomography (CT).

4. A method as recited in claim 1, wherein the first mode of measuring the living body is two-dimensional computer-aided full-dose X-ray tomography (CT).

5. A method as recited in claim 1, wherein the first mode of measuring the living body is two-dimensional nuclear magnetic resonance imaging (MRI).

6. A method as recited in claim 1, wherein the first mode of measuring the living body is two-dimensional positron emission tomography (PET) imaging.

7. A method as recited in claim 1, wherein the moving target tissue is a human prostate.

8. A method as recited in claim 1, wherein the moving target tissue is a human lung tumor.

9. A method as recited in claim 1, wherein the moving target tissue is a human liver lesion.

10. A method as recited in claim 1, said step of determining the elastic transform further comprising employing an elastic registration algorithm implemented at least in part in hardware, wherein said step of registering the high spatial resolution scan data to the high temporal resolution scan data is performed within a registration time interval short compared to an inverse of the first repeat rate.

11. A method as recited in claim 1, said step of receiving high spatial resolution data further comprising the steps of:
receiving first scan data based on the first mode of measuring the living body; and
preprocessing the first scan data to produce the high spatial resolution scan data with greater similarity to the high temporal resolution scan data than the first scan data.

12. A method as recited in claim 11, wherein:
said step of receiving high spatial resolution data further comprises the step of receiving boundary data that describes a boundary between the moving target tissue and other tissue in the living body; and
said step of preprocessing the first scan data further comprises preprocessing the first scan data based at least in part on the boundary data.

13. A method as recited in claim 12, said step of preprocessing the first scan data further comprising cropping the first data based at least in part on the boundary data to remove portions of the first scan data that correspond to portions of the living body that are not evident in the high temporal resolution scan data.

14. A method as recited in claim 12, said step of preprocessing the first scan data further comprising the steps of:
determining an average intensity value for all scan elements within the boundary; and
replacing the intensity values of all scan elements within the boundary with the average intensity value.

15. A method as recited in claim 12, said step of preprocessing the first scan data further comprising the step of applying an anisotropic diffusion filtering process for smoothing intensity values of scan elements.

16. A method as recited in claim 2, said step of receiving high temporal resolution scan data further comprising removing speckle in ultrasound data from the three-dimensional ultrasound second mode of measuring the living body.

17. A method as recited in claim 1, said step of determining the elastic transform further comprising using a sub-volume division based method for non-rigid spatial registration.

18. A method as recited in claim 1, said step of determining the elastic transform further comprising using a modified three dimensional Chain Mail algorithm to reduce folding artifacts.

19. A method as recited in claim 1, said step of determining the elastic transform further comprising maximizing a measure of mutual information.

20. A method as recited in claim 1, further comprising applying treatment to the target tissue based at least in part on the current spatial arrangement of the moving target tissue.

21. A method as recited in claim 20, said step of applying treatment to the target tissue based at least in part on the current spatial arrangement of the moving target tissue further comprising producing a three dimensional rendering of a volume in a vicinity of the target tissue.

22. A method as recited in claim 1, wherein the first mode measurement duration is before the treatment period of time, and the second mode measurement duration is during the treatment period of time.

23. A method as recited in claim 22, said step of indicating a current spatial arrangement of the moving target tissue further comprising indicating the current spatial arrangement of the moving target tissue during the treatment period of time.

24. A method as recited in claim 1, wherein the treatment is an intervention, such as a surgery or probe insertion.

25. A method for indicating disposition of moving target tissue in a living body, comprising:
receiving first scan data representing a scan of a living body based at least in part on a first mode of measuring the living body with high spatial resolution at a first measurement time;
receiving second scan data representing a scan of the living body based at least in part on a second mode of measuring the living body at a different second measurement time, wherein a moving target tissue in the living body changes from the first measurement time to the second measurement time in a way that is significantly different for treatment of the target tissue;
determining an elastic transform that registers the first scan data elastically to the second scan data; and
indicating a particular spatial arrangement of the moving target tissue in the living body at a particular time between the first measurement time and the second measurement time by interpolating the elastic transform.

26. A method as recited in claim 25, further comprising applying treatment to the target tissue based at least in part on the particular spatial arrangement of the moving target tissue.

27. A method as recited in claim 25, wherein the second mode is the same as the first mode.

28. A method as recited in claim 25, wherein the second mode and the first mode are computer aided X-ray tomography measurements.

29. A method as recited in claim 25, wherein the moving target tissue in the living body changes from the first measurement time to the second measurement time as part of a repeated physiological cycle.

30. An apparatus for indicating current disposition of moving target tissue in a living body, comprising:
means for receiving high spatial resolution scan data representing a scan of a living body based at least in part on a first mode of measuring the living body with high spatial resolution, wherein a repeat rate for repeatedly obtaining the high spatial resolution scan data based on the first mode of measuring the living body over a period of time for treating the living body is limited to be no greater than a first repeat rate;
means for receiving high temporal resolution scan data representing a scan of the living body based at least in part on a different second mode of measuring the living body over a second mode measurement duration, wherein a repeat rate for repeatedly obtaining the high temporal resolution scan data based on the second mode of measuring the living body over the period of time is greater than the first repeat rate;
means for determining an elastic transform that registers the high spatial resolution scan data elastically to the high temporal resolution scan data; and
means for indicating a current spatial arrangement of a moving target tissue in the living body during the second mode measurement time based on the elastic transform, wherein the moving target tissue changes over time among a plurality of spatial arrangements of the moving target tissue that are significantly different for treatment of the target tissue.

31. An apparatus for indicating disposition of moving target tissue in a living body, comprising:
means for receiving first scan data representing a scan of a living body based at least in part on a first mode of measuring the living body with high spatial resolution at a first measurement time;
means for receiving second scan data representing a scan of the living body based at least in part on a second mode of measuring the living body at a different second measurement time, wherein a moving target tissue in the living body changes from the first measurement time to the second measurement time in a way that is significantly different for treatment of the target tissue;
means for determining an elastic transform that registers the first scan data elastically to the second scan data; and
means for indicating a particular spatial arrangement of the moving target tissue in the living body at a particular time between the first measurement time and the second measurement time by interpolating the elastic transform.

32. A computer-readable medium carrying one or more sequences of instructions for indicating disposition of moving target tissue in a living body, wherein execution of the one or more sequences of instructions by one or more processors causes the one or more processors to perform the steps of:
receiving high spatial resolution scan data representing a scan of a living body based at least in part on a first mode of measuring the living body with high spatial resolution, wherein a repeat rate for repeatedly obtaining the high spatial resolution scan data based on the first mode of measuring the living body over a period of time for treating the living body is limited to be no greater than a first repeat rate;
receiving high temporal resolution scan data representing a scan of the living body based at least in part on a different second mode of measuring the living body over a second mode measurement duration, wherein a repeat rate for repeatedly obtaining the high temporal resolution scan data based on the second mode of measuring the living body over the period of time is greater than the first repeat rate;
determining an elastic transform that registers the high spatial resolution scan data elastically to the high temporal resolution scan data; and
indicating a current spatial arrangement of a moving target tissue in the living body during the second mode measurement duration based on the elastic transform, wherein the moving target tissue changes over time among a plurality of spatial arrangements of the moving target tissue that are significantly different for treatment of the target tissue.

33. A computer-readable medium carrying one or more sequences of instructions for indicating disposition of moving target tissue in a living body, wherein execution of the one or more sequences of instructions by one or more processors causes the one or more processors to perform the steps of:
receiving first scan data representing a scan of a living body based at least in part on a first mode of measuring the living body with high spatial resolution at a first measurement time;
receiving second scan data representing a scan of the living body based at least in part on a second mode of measuring the living body at a different second measurement time, wherein a moving target tissue in the living body changes from the first measurement time to the second measurement time in a way that is significantly different for treatment of the target tissue;
determining an elastic transform that registers the first scan data elastically to the second scan data; and
indicating a particular spatial arrangement of the moving target tissue in the living body at a particular time between the first measurement time and the second measurement time by interpolating the elastic transform.

34. An apparatus for indicating disposition of moving target tissue in a living body, comprising:
a logic circuit configured for determining an elastic transform that registers a first scan elastically to a second scan in a time short compared to a duration for a first mode of measuring the living body;
a processor;
a computer readable medium carrying one or more sequences of instructions, wherein execution of the one or more sequences of instructions by the processor causes the processor to perform the steps of
receiving high spatial resolution scan data representing a scan of a living body based at least in part on the first mode of measuring the living body with high spatial resolution, wherein a repeat rate for repeatedly obtaining the high spatial resolution scan data based on the first mode of measuring the living body over a period of time for treating the living body is limited to be no greater than a first repeat rate;
receiving high temporal resolution scan data representing a scan of the living body based at least in part on a different second mode of measuring the living body over a second mode measurement duration, wherein a repeat rate for repeatedly obtaining the high temporal resolution scan data based on the second mode of measuring the living body over the period of time is greater than the first repeat rate;
invoking the logic circuit for determining an elastic transform that registers the high spatial resolution scan data to the high temporal resolution scan data; and
indicating a current spatial arrangement of a moving target tissue in the living body during the second mode measurement duration, wherein the moving target tissue changes over time among a plurality of spatial arrangements of the moving target tissue that are significantly different for treatment of the target tissue.

35. An apparatus for indicating disposition of moving target tissue in a living body, comprising:
a logic circuit configured for determining an elastic transform that registers a first scan elastically to a second scan in a time short compared to a measurement duration for a first mode of measuring a living body;
a processor;
a computer readable medium carrying one or more sequences of instructions, wherein execution of the one or more sequences of instructions by the processor causes the processor to perform the steps of
receiving first scan data representing a scan of a living body based at least in part on a first mode of measuring the living body with high spatial resolution at a first measurement time;
receiving second scan data representing a scan of the living body based at least in part on a second mode of measuring the living body at a different second measurement time, wherein a moving target tissue in the living body changes from the first measurement time to the second measurement time in a way that is significantly different for treatment of the target tissue;
invoking the logic circuit to determine an elastic transform that registers the first scan data elastically to the second scan data; and
indicating a particular spatial arrangement of the moving target tissue in the living body at a particular time between the first measurement time and the second measurement time by interpolating the elastic transform.
